(19) United States
(12) Patent Application Publication
(10) Pub. No.: US 2003/0087287 A1
(43) Pub. Date: May 8, 2003

(54) Novel g-protein-coupled receptor protein and dna thereof

(76) Inventors: Masanori Miwa, Tsukuba-shi, Ibaraki (JP); Hideki Matsui, Tsukuba-shi, Ibaraki (JP); Yasushi Shintani, Osaka (JP)

Correspondence Address: TAKEDA PHARMACEUTICALS NORTH AMERICA, INC, INTELLECTUAL PROPERTY DEPARTMENT, 475 HALF DAY ROAD, SUITE 500, LINCOLNSHIRE, IL, 60069, US

(21) Appl. No.: 10/240,072

(22) PCT Filed: Mar. 27, 2001
(86) PCT No.: PCT/JP01/02446

(30) Foreign Application Priority Data
Mar. 28, 2000 (JP) 2000-092970

(51) Int. Cl. C12Q 1/68; G01N 33/53; G01N 33/567; C07H 21/04; C07K 14/705; C12P 21/02; C12N 5/06; A61K 38/17

(52) U.S. Cl. 435/6; 435/7.2; 435/69.1; 435/320.1; 435/325; 530/350; 530/388.22; 536/23.5; 514/12

(57) ABSTRACT

The human leukocyte-derived G protein-coupled receptor protein, a nucleic acid encoding the same and its derivative are useful in determining a ligand (an antagonist) to the receptor protein of the present invention, as an agent for the prevention and/or treatment of diseases associated with the dysfunction of the G protein-coupled receptor protein, etc.

### FIELD OF THE INVENTION

The present invention relates to a novel human leukocyte-derived G protein-coupled receptor protein and a DNA encoding the same.

### BACKGROUND ART

Most physiologically active substances such as hormones, neurotransmitters, etc. regulate the physiological functions through specific receptor proteins located in a cell membrane. Many of these receptor proteins mediate the transmission of intracellular signals via activation of guanine nucleotide-binding proteins (hereinafter sometimes merely referred to as G proteins) with which the receptor is coupled. These receptor proteins possess the common structure, i.e. seven transmembranes domains and are thus collectively referred to as G protein-coupled receptor proteins or seven-transmembrane receptor proteins (7TMR).

G protein-coupled receptor proteins exist on each functional cell surface of the cells and internal organs of a living body and play physiologically important roles as the targets of molecules, for example, hormones, neurotransmitters, physiologically active substances and the like, which molecules regulate the functions of these cells and organs in a living body. The receptors mediate signal transduction in a cell by binding to physiologically active substances and various reactions such as activation or suppression of cells are caused by the transduced signal.

To clarify the relation of substances that regulate complicated functions in cells or internal organs of various living bodies to their specific receptor proteins, in particular, to G protein-coupled receptor proteins, would elucidate the functional mechanisms of cells or internal organs in various living body and thus provide a very important means for development of drugs closely associated with such functions.

For example, in various organs of a living body, the physiological functions are controlled through regulation by many hormones, hormone-like substances, neurotransmitters or physiologically active substances. In particular, physiologically active substances present on various sites of a living body regulate their physiological functions through each of the corresponding receptor proteins. Still, there are many unknown hormones, neurotransmitters or other physiologically active substances exist in the body and only a few receptor proteins have been reported on their structures so far. In addition, many known receptor proteins yet remain unclear as to whether their subtypes exist.

It is also very important for development of pharmaceuticals to clarify the relation between substances that regulate complex functions in vivo and their specific receptor proteins. Furthermore, for efficient screening of agonists and antagonists to receptor proteins in development of pharmaceuticals, it was necessary to unravel the functions of receptor protein genes expressed in vivo and to express the genes in an appropriate expression system.

In recent years, a random analysis of cDNA sequences has been actively performed as a method for analyzing genes expressed in vivo. The sequences of cDNA fragments thus obtained have been registered and published to databases as Expressed Sequence Tag (EST). However, since most EST contains only the sequence information, it is difficult to predict the functions.

Substances that inhibit the binding of G protein-coupled receptors to physiological active substances (i.e., ligands) and substances that bind to physiologically active substances thereby to induce signal transductions similar to those induced by the physiologically active substances (i.e., ligands) have been used for pharmaceuticals as antagonists or agonists specific to the receptors for regulating the biological functions. Accordingly, it is very important to discover a new G protein-coupled receptor protein, which is not only important for physiological expression in vivo but can be a target for developing pharmaceuticals, and to clone the genes (e.g., cDNAs), in search for ligands, agonists and antagonists specific to the novel G protein-coupled receptor protein.

However, not all G protein-coupled receptors have been found. Even now, there are many unknown G protein-coupled receptors and the corresponding ligands are unidentified, that is, orphan receptors. It has thus been seriously awaited to explore a novel G protein-coupled receptor and clarify its function.

G protein-coupled receptors are useful in searching for novel physiologically active substances (i.e., ligands) using the signal transduction activity as an indicator and in searching for agonists and antagonists to the receptors. Even if no physiological ligand is found, agonists and antagonist to the receptors may be prepared by analyzing the physiological activity of the receptor through receptor inactivation experiments (knockout animal). Ligands, agonists, and antagonists to these receptors are expected to be used as prophylactic/therapeutic and diagnostic drugs for diseases associated with the dysfunction of G protein-coupled receptors.

In addition, hypofunction or hyperfunction of G protein-coupled receptors due to genetic variation in vivo causes some disorders in many cases. In this case, the receptors may be used not only for administration of antagonists or agonists to the receptors, but also for gene therapy by transfection of the receptor gene into the body (or some particular organs) or by transfection of an antisense nucleic acid to the receptor gene. In such a gene therapy, a base sequence of the receptor constitutes information essentially required to search deletion or mutation on the gene. The receptor gene is also applicable as a prophylactic/therapeutic and diagnostic drug for diseases associated with dysfunction of the receptor.

### DISCLOSURE OF THE INVENTION

The present invention provides a useful and novel G protein-coupled receptor protein as described above. That is, the present invention provides a novel G protein-coupled receptor protein, its partial peptides or salts thereof, as well as a polynucleotide (DNA and RNA, and derivatives thereof) containing the polynucleotide (DNA and RNA, and derivatives thereof) encoding the G protein-coupled receptor protein or and its partial peptide, a recombinant vector containing the polynucleotide, a transformant bearing the recombinant vector, methods for manufacturing the G protein-coupled receptor protein or salts thereof, antibodies to the G protein-coupled receptor protein, its partial peptides or salts thereof, compounds that alter the expression level of said G protein-coupled receptor protein, methods for determination of ligands to the G protein-coupled receptor protein, methods for screening compounds (antagonists and agonists) or salts thereof that alter the binding property of ligands and the G protein-coupled receptor protein, kits for use in the screening methods, compounds (antagonists and agonists) or salts thereof that alter the binding property of ligands and the G protein-coupled receptor protein, which are obtainable by the screening methods or the screening kits, pharmaceutical compositions comprising the compounds (antagonists and agonists) that alter the binding property of ligands to the G protein-coupled receptor protein or compounds that alter the expression level of the G protein-coupled receptor protein, or salts thereof, and the like.

The inventors performed extensive studies and as a result, succeeded in isolating a cDNA encoding a human leukocyte-derived novel G protein-coupled receptor protein and in analyzing the entire base sequence of the cDNA. The amino acid sequence deduced from the base sequence has supported that the first to the seventh transmembrane domains were observed on the hydrophobic plotting analysis, confirming that the protein encoded by the cDNA is a 7 transmembrane type G protein-coupled receptor protein. Based on these findings, the present inventors further proceeded the investigations and as a result, have come to accomplish the present invention.

Thus, the present invention relates to:

(1) A G protein-coupled receptor protein containing the same or substantially the same amino acid sequence as the amino acid sequence shown by SEQ ID NO: 1, or a salt thereof;

(2) A partial peptide of the G protein-coupled receptor protein according to (1), or a salt thereof;

(3) A polynucleotide containing a polynucleotide encoding the G protein-coupled receptor protein according to (1) or the partial peptide according to (2);

(4) The polynucleotide according to (4), which is a DNA;

(5) The polynucleotide according to (3), which has the base sequence represented by SEQ ID NO: 2 or SEQ ID NO: 3;

(6) A recombinant vector containing the polynucleotide according to (3);

(7) A transformant transformed by the recombinant vector according to (6);

(8) A method of manufacturing the G protein-coupled receptor protein according to (1) or the partial peptide according to (2), or a salt thereof, which comprises culturing the transformant according to (7) and producing the G protein-coupled receptor protein according to (1) or the partial peptide according to (2);

(9) An antibody to the G protein-coupled receptor protein according to (1) or the partial peptide according to (2), or a salt thereof;

(10) The Antibody according to (9), which is a neutralizing antibody to inactivate signal transduction of the G protein-coupled receptor protein according to (1);

(11) A diagnostic composition comprising the antibody according to (9);

(12) A ligand to the 6 protein-coupled receptor protein or its salt according to (1), which is obtainable using the G protein-coupled receptor protein according to (1) or the partial peptide according to (2), or a salt thereof;

(13) A pharmaceutical composition comprising the ligand to the G protein-coupled receptor protein according to (12);

(14) A method of determining a ligand to the G protein-coupled receptor protein or its salt according to (1), wherein the G protein-coupled receptor protein according to (1) or the partial peptide according to (2), or a salt thereof is used;

(15) A method of screening a compound or its salt that alters the binding property between a ligand and the G protein-coupled receptor protein or its salt according to (1), which comprises using the G protein-coupled receptor protein according to (1) or the partial peptide according to (2), or a salt thereof;

(16) A kit for screening a compound or its salt that alters the binding property between a ligand and the G protein-coupled receptor protein or its salt according to (1), comprising the G protein-coupled receptor protein according to (1) or the partial peptide according to (2), or a salt thereof;

(17) A compound or its salt that alters the binding property between a ligand and the G protein-coupled receptor protein or its salt according to (1), which is obtainable using the screening method according to (15) or the screening kit according to (16);

(18) A pharmaceutical composition comprising the compound or its salt that alters the binding property between a ligand and the G protein-coupled receptor protein or its salt according to (1), which is obtainable using the screening method according to (15) or the screening kit according to (16);

(19) A polynucleotide hybridizable to the polynucleotide according to (3) under high stringent conditions.

(20) A polynucleotide comprising a base sequence complementary to the polynucleotide according to (3), or a part thereof.

(21) A method of quantifying mRNA of the G protein-coupled receptor protein according to (1), which comprises using the polynucleotide according to (3), or a part thereof;

(22) A method of quantifying the G protein-coupled receptor protein according to (1), which comprises using the antibody according to (9);

(23) A diagnostic agent for diseases associated with the function of the G protein-coupled receptor protein according to (1), which comprises using the quantifying method according to (21) or (22);

(24) A method of screening a compound or its salt that alters the expression level of the G protein-coupled receptor protein according to (1), which comprises using the quantifying method according to (21);

(25) A method of screening a compound or its salt that alters the amount of the G protein-coupled receptor protein according to (1) on a cell membrane, which comprises using the quantifying method according to (22);

(26) A compound or its salt that alters the expression level of the G protein-coupled receptor protein according to (1), which is obtainable using the screening method according to (24);

(27) A compound or its salt that alters the amount of the G protein-coupled receptor protein according to (1) on a cell membrane, which is obtainable using the screening method according to (25); and the like.

The present invention further provides:

(28) The G protein-coupled receptor protein or its salt according to (1), wherein the protein is a protein containing: (i) an amino acid sequence represented by SEQ ID NO: 1, in which 1, 2 or more amino acids (preferably about 1 to about 30 amino acids, more preferably about 1 to about 9 amino acids, most preferably several (1 to 5) amino acids) are deleted; (ii) an amino acid sequence represented by SEQ ID NO: 1, to which 1, 2 or more amino acids (preferably about 1 to about 30 amino acids, more preferably about 1 to about 10 amino acids, most preferably several (1 to 5) amino acids) are added; (iii) an amino acid sequence represented by SEQ ID NO: 1, in which 1, 2 or more amino acids (preferably about 1 to about 30 amino acids, more preferably about 1 to about 10 amino acids, most preferably several (1 to 5) amino acids) are substituted by other amino acids; or (iv) a combination of the above amino acid sequences;

(29) The method of determining a ligand according to (14), which comprises contacting the G protein-coupled receptor protein or its salt according to (1) or the partial peptide or its salt according to (2) with a test compound;

(30) The method of determining a ligand according to (29), wherein the ligand is, e.g., angiotensin, bombesin, canavinoid, cholecystokinin, glutamine, serotonin, melatonin, neuropeptide Y, opioid, purines, vasopressin, oxytocin, PACAP, secretin, glucagon, calcitonin, adrenomedulin, somatostatin, GHRH, CRF, ACTH, GRP, PTH, VIP (vasoactive intestinal polypeptide), somatostatin, dopamine, motilin, amylin, bradykinin, CGRP (calcitonin gene-related peptide), leukotrienes, pancreastatin, prostaglandins, thromboxane, adenosine, adrenaline, a and β-chemokines (e.g., IL-8, GROα, GROβ, GROγ, NAP-2, ENA-78, PF4, IP10, GCP-2, MCP-1, HC14, MCP-3, I-309, MIP1α, MIP-1β, RANTES, etc.), endothelin, enterogastrin, histamine, neurotensin, TRH, pancreatic polypeptide or galanin, lysophosphatidic acid (LPA) or sphingosine-1-phosphate;

(31) The method of screening according to (15), wherein comparison is made between (i) the case in which the G protein-coupled receptor protein or its salt according to (1) or the partial peptide or its salt according to (2) is brought in contact with a ligand and (ii) the case in which the G protein-coupled receptor protein or its salt according to (1) or the partial peptide or its salt according to (2) is brought in contact with a ligand and a test compound;

(32) A method of screening a compound or its salt that alters the binding property between a ligand and the G protein-coupled receptor protein or its salt according to (1), which comprises measuring an amount of the labeled ligand bound to the G protein-coupled receptor protein or its salt according to (1) or the partial peptide or its salt according to (2), (i) when the labeled ligand is brought in contact with the G protein-coupled receptor protein or its salt according to (1) or the partial peptide or its salt according to (2), and (ii) when the labeled ligand and a test compound are brought in contact with the G protein-coupled receptor protein or its salt according to (1) or the partial peptide or its salt according to (2); and comparing the amounts of (i) and (ii);

(33) A method of screening a compound or its salt that alters the amount of a ligand bound to a cell containing the G protein-coupled receptor protein according to (1), which comprises measuring an amount of the labeled ligand bound to the cell (i) when the labeled ligand is brought in contact with the cell, and (ii) when the labeled ligand and a test compound are brought in contact with the cell, and comparing the amounts of (i) and (ii);

(34) A method of screening a compound or its salt that alters the binding property between a labeled ligand and a cell membrane fraction containing the G protein-coupled receptor protein according to (1), which comprises measuring an amount of the labeled ligand bound to the cell membrane fraction (i) when the labeled ligand is brought in contact with the cell membrane fraction, and (ii) when the labeled ligand and a test compound are brought in contact with the cell membrane fraction, and comparing the amounts of (i) and (ii);

(35) A method of screening a compound or its salt that alters the binding property between a ligand and the G protein-coupled receptor protein or its salt according to (1), which comprises measuring an amount of the labeled ligand bound to the G protein-coupled receptor protein expressed on a cell membrane of the transformant according to (7) by culturing the transformant, (i) when the labeled ligand is brought in contact with the G protein-coupled receptor protein expressed on a cell membrane of the transformant according to (7) by culturing the transformant, and (ii) when the labeled ligand and a test compound are brought in contact with the G protein-coupled receptor protein expressed, and comparing the amounts of (i) and (ii);

(36) A method of screening a compound or its salt that alters the binding property between a ligand and the G protein-coupled receptor protein or its salt according to (1), which comprises measuring a G protein-coupled receptor protein-mediated cell stimulating activity (i) when a compound that activates the G protein-coupled receptor protein or its salt according to (1) is brought in contact with a cell containing the G protein-coupled receptor protein according to (1) and (ii) when a compound that activates the G protein-coupled receptor protein or its salt according to (1) and a test compound are brought in contact with the cell containing the G protein-coupled receptor protein according to (1), and comparing the activities of (i) and (ii);

(37) A method of screening a compound or its salt that alters the binding property between a ligand and the G protein-coupled receptor protein or its salt according to (1), which comprises measuring a G protein-coupled receptor protein-mediated cell stimulating activity (i) when a compound that activates the G protein-coupled receptor protein or its salt according to (1) is brought in contact with a G protein-coupled receptor protein expressed on a cell membrane of the transformant according to (7) by culturing the transformant and (ii) when a compound that activates the G protein-coupled receptor protein or its salt according to (1) and a test compound are brought in contact with a G protein-coupled receptor protein expressed on a cell membrane of the transformant according to (7) by culturing the transformant, and comparing (i) and (ii);

(38) A method of screening according to (36) or (37), wherein the compound that activates the G protein-coupled receptor protein according to (1) is angiotensin, bombesin, canavinoid, cholecystokinin, glutamine, serotonin, melatonin, neuropeptide Y, opioid, purines, vasopressin, oxytocin, PACAP, secretin, glucagon, calcitonin, adrenomedulin, somatostatin, GHRH, CRF, ACTH, GRP, PTH, VIP (vasoactive intestinal polypeptide), somatostatin, dopamine, motilin, amylin, bradykinin, CGRP (calcitonin gene-related peptide), leukotrienes, pancreastatin, prostaglandins, thromboxane, adenosine, adrenaline, α and β -chemokines (e.g., IL-8, GROα, GROβ, GROγ, NAP-2, ENA-78, PF4, IP10, GCP-2, MCP-1, HC14, MCP-3, I-309, MIP1α, MIP-1β, RANTES, etc.), endothelin, enterogastrin, histamine, neurotensin, TRH, pancreatic polypeptide or galanin, lysophosphatidic acid (LPA) or sphingosine-1-phosphate;

(39) A compound or its salt that alters the binding property between a ligand and the G protein-coupled receptor or its salt according to (1), which is obtainable by the screening method of according to (31) through (38);

(40) A pharmaceutical composition comprising a compound or its salt that alters the binding property between a ligand and the G protein-coupled receptor protein or its salt according to (1), which is obtainable by screening method of according to (31) through (38);

(41) The screening kit according to (16), comprising a cell containing the G protein-coupled receptor protein according to (1);

(42) The screening kit according to (16), comprising a cell membrane fraction containing the G protein-coupled receptor protein according to (1);

(43) The screening kit according to (16), comprising a G protein-coupled receptor protein expressed on a cell membrane of the transformant according to (8) by culturing the transformant;

(44) A compound or its salt that alters the binding property between a ligand and the G protein-coupled receptor protein or its salt according to (1), which is obtainable using the screening kit according to (41) through (43);

(45) A pharmaceutical composition comprising a compound or its salt that alters the binding property between a ligand and the G protein-coupled receptor protein or its salt according to (1), which is obtainable screening kit according to (41) through (43);

(46) A method of quantifying the G protein-coupled receptor protein or its salt according to (1), or the partial peptide or its salt according to (2), which comprises contacting the antibody according to (9) with the G protein-coupled receptor protein or its salt according to (1) or partial peptide or its salt according to (2);

(47) A method of quantifying the G protein-coupled receptor protein or its salt according to (1), or the partial peptide or its salt according to (2), in a test sample, which comprises competitively reacting the antibody according to (9) with a test sample and a labeled form of the G protein-coupled receptor protein or its salt according to (1) or a labeled form of the partial peptide or its salt according to (2) and, measuring a ratio of the labeled G protein-coupled receptor protein or its salt according to (1) or the labeled partial peptide or its salt according to (2), which is bound to the antibody; and,

(48) A method of quantifying the G protein-coupled receptor protein or its salt according to (1) or the partial peptide or its salt according to (2), in a test sample, which comprises reacting a test sample simultaneously or sequentially with the antibody according to (9) immobilized on a carrier and a labeled form of the antibody according to (9), and then assaying the activity of a labeling agent on the immobilized carrier.

### BRIEF DESCRIPTION OF THE DRAWINGS

FIG. 1 shows the base sequence of cDNA (hTGR3T) encoding human leukocyte-derived novel G protein-coupled receptor protein hTGR3 of the present invention obtained in EXAMPLE 1, and the amino acid sequence deduced therefrom (continued to FIG. 2).

FIG. 2 shows the base sequence of cDNA (hTGR3T) encoding human leukocyte-derived novel G protein-coupled receptor protein hTGR3 of the present invention obtained in EXAMPLE 1, and the amino acid sequence deduced therefrom (continued from FIG. 1).

FIG. 3 shows the base sequence of cDNA (hTGR3T) encoding human leukocyte-derived novel G protein-coupled receptor protein hTGR3 of the present invention obtained in EXAMPLE 1, and the amino acid sequence deduced therefrom (continued to FIG. 4).

FIG. 4 shows the base sequence of cDNA (hTGR3T) encoding human leukocyte-derived novel G protein-coupled receptor protein hTGR3 of the present invention obtained in EXAMPLE 1, and the amino acid sequence deduced therefrom (continued from FIG. 3).

FIG. 5 shows the hydrophobic plotting of human leukocyte-derived novel G protein-coupled receptor protein hTGR3 of the present invention.

FIG. 6 shows the analytical results of distribution of TGR3 expressed in human tissues, tested in EXAMPLE 2.

### BEST MODE FOR CARRYING OUT THE INVENTION

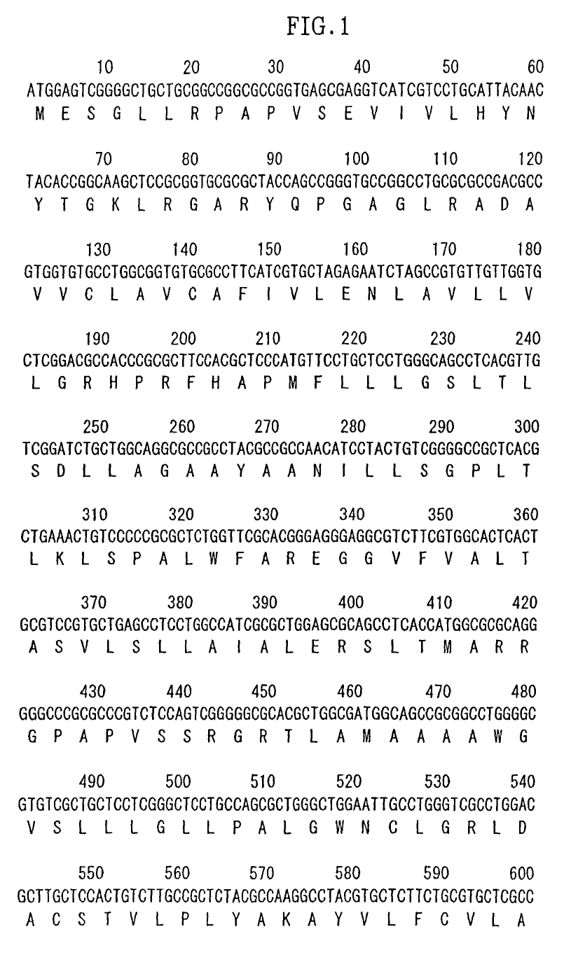

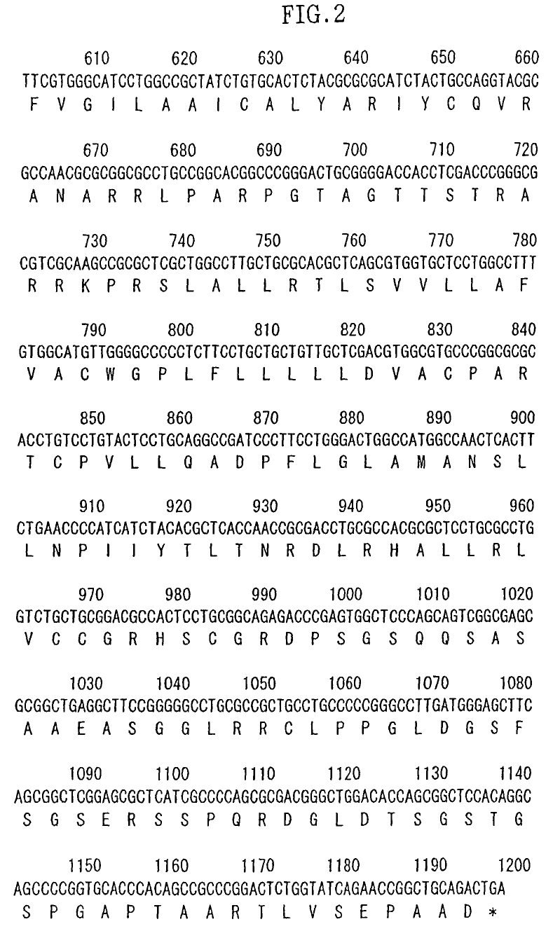

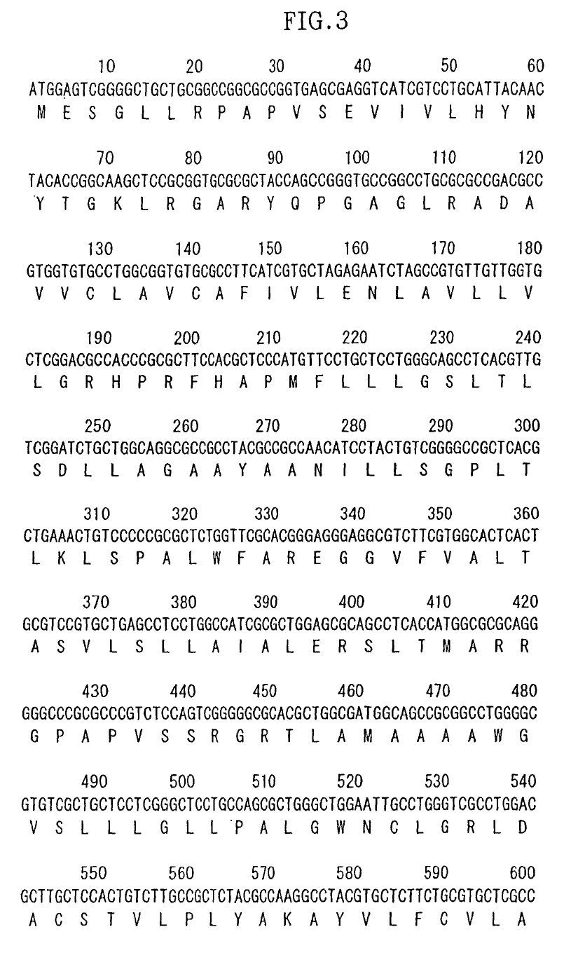

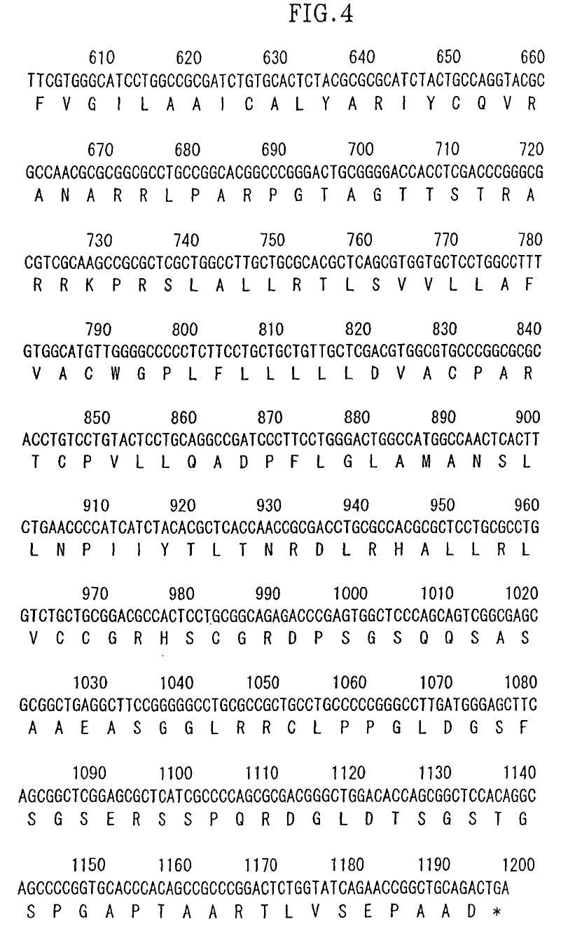

The G protein-coupled receptor protein of the present invention (hereinafter sometimes simply referred to as the receptor protein) is the receptor protein containing the same or substantially the same amino acid sequence as the amino acid sequence represented by SEQ ID NO: 1 (the amino acid sequence shown by FIGS. 1 through 4).

The receptor protein of the present invention may be any protein derived from any cells of human and another mammal (e.g., guinea pig, rat, mouse, rabbit, swine, sheep, bovine, monkey, etc.), for example, spleen cells, nerve cells, glial cells, P cells of pancreas, bone marrow cells, mesangial cells, Langerhans' cells, epidermal cells, epithelial cells, endothelial cells, fibroblasts, fibrocytes, muscular cells, fat cells, immunocytes (e.g., macrophage, T cells, B cells, natural killer cells, mast cells, neutrophils, basophils, eosinophils or monocytes), megakaryocytes, synovial cells, chondrocytes, osteocytes, osteoblasts, osteoclasts, mammary gland cells, hepatocytes or interstitial cells, or precursor cells, stem cells or cancer cells of these cells, and the like; hemocytes; or any tissues containing such cells, for example, brain or various parts of the brain (e.g., olfactory bulb, amygdala, cerebral basal ganglia, hippocampus, thalamus, hypothalamus, substhanlamic nucleus, cerebral cortex, medulla oblongata, cerebellum, occipital pole, frontal lobe, temporal lobe, putamen, caudate nucleus, corpus callosum, substantia nigra), spinal cord, pituitary, stomach, pancreas, kidney, liver, genital gland, thyroid gland, gallbladder, bone marrow, adrenal gland, skin, muscle, lung, digestive tract (e.g., large intestine, small intestine), blood vessel, heart, thymus, spleen, submandibular gland, peripheral blood, peripheral hemocyte, prostate, testicle, testis, ovary, placenta, uterus, bone, joint, skeletal muscle and the like (in particular, brain and various parts of the brain). The receptor protein may also be synthetic.

The amino acid sequence which has the same or substantially the same as the amino acid sequence represented by SEQ ID NO: 1. includes an amino acid sequence having at least about 90% homology, preferably at least about 95% homology, more preferably at least about 98% homology, much more preferably at least about 99% homology, most preferably at least about 99.5% homology, to the amino acid sequence represented by SEQ ID NO: 1.

A preferred example of the protein containing substantially the same amino acid sequence as the amino acid sequence represented by SEQ ID NO: 1 is a protein containing substantially the same amino acid sequence as the amino acid sequence represented by SEQ ID NO: 1 and having an activity substantially equivalent to that of the amino acid sequence represented by SEQ ID NO: 1.

As the substantially equivalent activity, there are, for example, a ligand binding activity, a signal transduction activity, and the like. The term substantially equivalent is used to mean that these activities are equivalent in nature to one another. It is thus preferred that the activity such as a ligand binding activity or a signal transduction activity is equivalent (e.g., approximately 0.01 to 100 times, preferably about 0.5 to 20 times, more preferably about 0.5 to 2 times), but quantitative factors such as the degree of these activities, a molecular weight of protein, etc. may be different from each other.

The activities such as a ligand binding activity, a signal transduction activity, etc. may be determined by modifications of publicly known methods, for example, by the methods of determining ligands or the screening methods, which will be later described.

As the receptor protein of the present invention, there may be used a protein containing (i) an amino acid sequence represented by SEQ ID NO: 1, in which 1, 2 or more amino acids (preferably about 1 to about 30 amino acids, more preferably about 1 to about 10 amino acids, most preferably several (1 to 5) amino acids) are deleted; (ii) an amino acid sequence represented by SEQ ID NO: 1, to which 1, 2 or more amino acids (preferably about 1 to about 30 amino acids, more preferably about 1 to about 10 amino acids, most preferably several (1 to 5) are added; (iii) an amino acid sequence represented by SEQ ID NO: 1, in which 1, 2 or more amino acids (preferably about 1 to about 30 amino acids, more preferably about 1 to about 10 amino acids, most preferably several (1 to 5) are substituted by other amino acids; and (iv) a combination of the amino acid sequences above.

The receptor proteins in the present specification are designated according to the established practice of describing peptides, in which the left end is the N-terminal (amino terminal) and the right end is the C-terminal (carboxyl terminal). In the receptor proteins of the present invention including the receptor protein containing the amino acid sequence represented by SEQ ID No: 1, the C-terminal is normally a carboxyl group (—COOH) or a carboxylate (—COO⁻), but the C-terminal may be an amide (—CONH₂) or an ester (—COOR).

Herein, examples of the ester group shown by R include a C₁₋₆ alkyl group such as methyl, ethyl, n-propyl, isopropyl, n-butyl, etc.; a C₃₋₈ cycloalkyl group such as cyclopentyl, cyclohexyl, etc.; a C₆₋₁₂ aryl group such as phenyl, α-naphthyl, etc.; a C₇₋₁₄ aralkyl group such as a phenyl-C₁₋₂ alkyl group, e.g., benzyl, phenethyl, etc.; an α-naphthyl-C₁₋₂ alkyl group, e.g., α-naphthylmethyl, etc.; and the like. In addition, pivaloyloxymethyl or the like which is used widely as an ester for oral administration may also be used.

Where the receptor protein of the present invention contains a carboxyl group (or carboxylate) at a position other than the C-terminal, it may be amidated or esterified and such an amide or ester is also included within the receptor protein of the present invention. The ester group may be the same group as that described with respect to the above C-terminal.

Further, the receptor protein of the present invention includes derivatives wherein the amino group of N-terminal methionine residue of the above protein is protected with a protecting group (e.g., an acyl group having 1 to 6 carbon atoms such as formyl group, acetyl group, etc.); those wherein the N-terminal region is cleaved in vivo and the glutamyl group formed is pyroglutaminated; and those wherein a substituent (e.g., —OH, —SH, —COOH, amino group, imidazole group, indole group, guanidino group, etc.) on the side chains of an amino acid in the molecule of the protein is protected with an appropriate protecting group (e.g., an acyl group having 1 to 6 carbon atoms such as an alkanoyl group having 2 to 6 carbon atoms, e.g., formyl group, acetyl group, etc.), or conjugated proteins such as glycoproteins having sugar chains.

Specific examples of the receptor protein of the present invention that can be used include a receptor protein containing the amino acid sequence shown by SEQ ID NO: 1, and the like.

The partial peptide of the receptor protein of the present invention (hereinafter sometimes merely referred to as the partial peptide) may be any peptide, as long as it is a partial peptide of the receptor protein of the present invention described above. For example, among the receptor protein molecules of the present invention, the region exposed to the outside of the cell membrane which has substantially the same ligand-binding activity, or the like, may be employed.

The partial peptide of the receptor protein of the present invention (hereinafter sometimes referred to as the partial peptide) may be any partial peptide, so long as it is a partial peptide of the receptor protein of the present invention described above. For example, in the receptor protein molecule of the present invention, those having the site exposed to the outside of a cell membrane, or the like may be used, so long as they retain substantially the same ligand binding activity.

An example of the partial peptide of the receptor protein containing the amino acid sequence represented by SEQ ID NO: 1 includes a peptide containing a domain which is analyzed to be an extracellular region (hydrophilic domain) by the hydrophobic plotting analysis. A peptide containing a hydrophobic site as a part of it may be used as well. A peptide containing the respective domains independently may also be used, although the partial peptide containing a plurality of domains at the same time may be used as well.

The partial peptides of the present invention are preferably those having the number of amino acids in the partial peptides of at least 20, preferably 50 or more, more preferably 100 or more, in terms of the amino acid sequence that constitutes the receptor protein of the present invention described above.

The term substantially the same amino acid sequence is used to mean an amino acid sequence having at least about 90% homology, preferably at least about 95% homology, more preferably at least about 98% homology, much more preferably at least about 99% homology, most preferably at least about 99.5% homology.

Herein, the term “substantially equivalent ligand binding activity” has the same definition as described above. The “substantially equivalent ligand binding activity” can be assayed in the same way as described above.

The partial peptides of the present invention may be those wherein 1, 2 or more amino acids (preferably approximately 1 to 10 amino acids, more preferably several (1 to 5) amino acids) may be deleted in the amino acid sequence described above; 1, 2 or more amino acids (preferably approximately 1 to 10 amino acids, more preferably several (1 to 5) amino acids) may be added to the amino acid sequence; or, 1, 2 or more amino acids (preferably approximately 1 to 10 amino acids, more preferably several, most preferably approximately 1 to 5) amino acids) may be substituted by other amino acids in the amino acid sequence.

In the partial peptides of the present invention, the C-terminal is normally a carboxyl group (—COOH) or a carboxylate (—COO⁻) but the C-terminal may be an amide (—CONH₂) or an ester (—COOR), as has been described with respect to the protein of the present invention.

As in the receptor protein of the present invention described above, the partial peptide of the present invention also includes those wherein the amino group of the N-terminal methionine residue is protected by a protecting group, those wherein the N-terminal residue is cleaved in vivo and the produced Gln is converted into pyroglutamate, those wherein substituents on the side chains of amino acids in the molecule are protected by appropriate protecting groups and conjugated peptides to which sugar chains are bound, that is glycopeptides, and the like.

In the partial peptides of the present invention, the C-terminal is normally a carboxyl group (—COOH) or a carboxylate (—COO⁻) but the C-terminal may be an amide (—CONH₂) or an ester (—COOR), as has been described with respect to the protein of the present invention.

The salts of the receptor protein or its partial peptide of the present invention include physiologically acceptable salts with acids or bases, and in particular, physiologically acceptable acid addition salts are preferred. Examples of such salts are salts with inorganic acids (e.g., hydrochloric acid, phosphoric acid, hydrobromic acid, sulfuric acid), salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid) and the like.

The receptor protein of the present invention or salts thereof may be manufactured by publicly known methods for purification of receptor proteins from the human or other mammalian cells or tissues described above. Alternatively, the receptor protein of the present invention or salts thereof may also be manufactured by culturing a transformant containing a DNA encoding the receptor protein of the present invention, as will be later described. Furthermore, the receptor protein of the present invention or salts thereof may also be manufactured by the method for protein synthesis, which will also be described hereinafter, or by modifications of such a method.

When the receptor protein or salts thereof are manufactured from human or mammalian tissues or cells, the human or mammalian tissues or cells are homogenized and extracted with an acid or the like, and the extract is isolated and purified by a combination of chromatography techniques such as reversed phase chromatography, ion exchange chromatography, and the like.

To synthesize the receptor protein or its partial peptide of the present invention, or salts or amides thereof, commercially available resins that are used for protein synthesis can be used. Examples of such resins include chloromethyl resin, hydroxymethyl resin, benzhydrylamine resin, aminomethyl resin, 4-benzyloxybenzyl alcohol resin, 4-methylbenzhydrylamine resin, PAM resin, 4-hydroxymethylmehtylphenyl acetamidomethyl resin, polyacrylamide resin, 4-(2′,4′-dimethoxyphenyl-hydroxymethyl)phenoxy resin, 4-(2′,4′-dimethoxyphenyl-Fmoc-aminoethyl) phenoxy resin, etc. Using these resins, amino acids wherein α-amino groups and functional groups on the side chains are appropriately protected are condensed on the resin in the order of the amino acid sequence of the objective protein or peptide, following various condensation methods publicly known in the art. At the end of the reaction, the protein is excised from the resin and at the same time, the protecting groups are removed. Then, intramolecular disulfide bond-forming reaction is performed in a highly diluted solution to obtain the objective protein or amides thereof.

For condensation of the protected amino acids described above, a variety of activation reagents usable for protein synthesis may be employed, but carbodiimides are particularly preferably used. Examples of such carbodiimides include DCC, N,N′-diisopropylcarbodiimide, N-ethyl-N′-(3-dimethylaminopropyl)carbodiimide, etc. For activation by these reagents, the protected amino acids are added directly to the resin together with a racemization inhibitor (e.g., HOBt, HOOBt), or the protected amino acids are previously activated in the form of symmetric acid anhydrides, HOBt esters or HOOBt esters, followed by adding the thus activated protected amino acids to the resin.

Solvents suitable for use in the activation of protected amino acids or in the condensation with resins may be selected from solvents that are known to be usable for protein condensation reactions. Examples of such solvents are acid amides such as N,N-dimethylformamide, N,N-dimethylacetamide, N-methylpyrrolidone, etc.; halogenated hydrocarbons such as methylene chloride, chloroform, etc.; alcohols such as trifluoroethanol, etc.; sulfoxides such as dimethylsulfoxide, etc.; ethers such as pyridine, dioxane, tetrahydrofuran, etc.; nitrites such as acetonitrile, propionitrile, etc.; esters such as methyl acetate, ethyl acetate, etc.; or appropriate mixtures of these solvents, and the like. The reaction temperature is appropriately chosen from the range known to be applicable to protein bond forming reactions and is usually selected from the range of approximately −20° C. to 50° C. The activated amino acid derivatives are used generally in an excess of 1.5 to 4 times. The condensation is examined using the ninhydrin reaction; when the condensation is insufficient, the condensation can be completed by repeating the condensation reaction without removal of the protecting groups. When the condensation is yet insufficient even after repeating the reaction, unreacted amino acids are acetylated with acetic anhydride or acetylimidazole to cancel any possible adverse affect on the subsequent reaction.

Examples of the protecting groups used to protect the starting amino groups include Z, Boc, tertiary-pentyloxycarbonyl, isobornyloxycarbonyl, 4-methoxybenzyloxycarbonyl, Cl-Z, Br-Z, adamantyloxycarbonyl, trifluoroacetyl, phthaloyl, formyl, 2-nitrophenylsulphenyl, diphenylphosphinothioyl, Fmoc, etc.

A carboxyl group can be protected by, e.g., alkyl esterification (in the form of linear, branched or cyclic alkyl esters of methyl, ethyl, propyl, butyl, t-butyl, cyclopentyl, cyclohexyl, cycloheptyl, cyclooctyl, 2-adamantyl, etc.), aralkyl esterification (e.g., benzyl ester, 4-nitrobenzyl ester, 4-methoxybenzyl ester, 4-chlorobenzyl ester, and benzhydryl ester), phenacyl esterification, benzyloxycarbonyl hydrazidation, t-butoxycarbonyl hydrazidation, trityl hydrazidation, etc.

The hydroxyl group of serine can be protected by, for example, its esterification or etherification. Examples of groups appropriately used for the esterification include a lower alkanoyl group such as acetyl group, etc., an aroyl group such as benzoyl group, etc., a group derived from carbonic acid such as benzyloxycarbonyl group, ethoxycarbonyl group, etc. Examples of a group appropriately used for the etherification include benzyl group, tetrahydropyranyl group, t-butyl group, etc.

Examples of groups for protecting the phenolic hydroxyl group of tyrosine include Bzl, Cl₂-Bzl, 2-nitrobenzyl, Br-Z, t-butyl, etc.

Examples of groups used to protect the imidazole moiety of histidine include Tos, 4-methoxy-2,3,6-trimethylbenzenesulfonyl, DNP, benzyloxymethyl, Bum, Boc, Trt, Fmoc, etc.

Examples of the activated carboxyl groups in the starting amino acids include the corresponding acid anhydrides, azides, activated esters [esters with alcohols (e.g., pentachlorophenol, 2,4,5-trichlorophenol, 2,4-dinitrophenol, cyanomethyl alcohol, p-nitrophenol, HONB, N-hydroxysuccirnide, N-hydroxyphthalimide, HOBt], etc. As the activated amino acids in which the amino groups are activated in the starting material, the corresponding phosphoric amides are employed.

To eliminate (split off) the protecting groups, there are used catalytic reduction under hydrogen gas flow in the presence of a catalyst such as Pd-black, Pd-carbon, etc.; an acid treatment with anhydrous hydrogen fluoride, methanesulfonic acid, trifluoromethanesulfonic acid or trifluoroacetic acid, or a mixture solution of these acids, etc.; a treatment with a base such as diisopropylethylamine, triethylamine, piperidine, piperazine, etc.; reduction with sodium in liquid ammonia, or the like. The elimination reaction of the protecting group by the acid treatment described above is carried out generally at a temperature of approximately −20° C. to 40° C. In the acid treatment, it is efficient to add a cation scavenger such as anisole, phenol, thioanisole, m-cresol, p-cresol, dimethylsulfide, 1,4-butanedithiol, 1,2-ethanedithiol, etc. Furthermore, 2,4-dinitrophenyl group known as the protecting group for the imidazole of histidine is removed by a treatment with thiophenol. Formyl group used as the protecting group of the indole of tryptophan is eliminated by the aforesaid acid treatment in the presence of 1,2-ethanedithiol, 1,4-butanedithiol, etc. as well as by a treatment with an alkali such as a dilute sodium hydroxide solution, dilute ammonia, etc.

Protection of functional groups that should not be involved in the reaction of the starting materials, protecting groups, elimination of the protecting groups and activation of functional groups involved in the reaction may be appropriately chosen from publicly known groups and publicly known means.

In another method for obtaining the amidated protein, for example, the α-carboxyl group of the carboxy terminal amino acid is first protected by amidation;

the peptide (protein) chain is then extended from the amino group side to a desired length. Thereafter, a protein in which only the protecting group of the N-terminal α-amino group has been eliminated from the peptide and a protein in which only the protecting group of the C-terminal carboxyl group has been eliminated are manufactured. The two proteins are condensed in a mixture of the solvents described above. The details of the condensation reaction are the same as described above. After the protected protein obtained by the condensation is purified, all the protecting groups are eliminated by the method described above to give the desired crude protein. This crude protein is purified by various known purification means. Lyophilization of the major fraction gives the amide of the desired protein.

To prepare the esterified protein, for example, the α-carboxyl group of the carboxy terminal amino acid is condensed with a desired alcohol to prepare the amino acid ester, which is followed by procedure similar to the preparation of the amidated protein above to give the desired esterified protein.

The partial peptide of the protein of the present invention or salts thereof can be manufactured by publicly known methods for peptide synthesis, or by cleaving the protein of the present invention with an appropriate peptidase. For the methods for peptide synthesis, for example, either solid phase synthesis or liquid phase synthesis may be used. That is, the partial peptide or amino acids that can construct the protein of the present invention are condensed with the remaining part of the protein of the present invention. Where the product contains protecting groups, these protecting groups are removed to give the desired peptide. Publicly known methods for condensation and elimination of the protecting groups are described in 1)-5) below.

1) M. Bodanszky & M. A. Ondetti: Peptide Synthesis, Interscience Publishers, New York (1966)

2) Schroeder & Luebke: The Peptide, Academic Press, New York (1965)

3) Nobuo Izumiya, et al.: Peptide Gosei-no-Kiso to Jikken (Basics and experiments of peptide synthesis), published by Maruzen Co. (1975)

4) Haruaki Yajima & Shunpei Sakakibara: Seikagaku Jikken Koza (Biochemical Experiment) 1, Tanpakushitsu no Kagaku (Chemistry of Proteins) IV, 205 (1977)

5) Haruaki Yajima, ed.: Zoku Iyakuhin no Kaihatsu (A sequel to Development of Pharmaceuticals), Vol. 14, Peptide Synthesis, published by Hirokawa Shoten

After completion of the reaction, the product may be purified and isolated by a combination of conventional purification methods such as solvent extraction, distillation, column chromatography, liquid chromatography, recrystallization and the like to give the partial peptide of the present invention. When the partial peptide obtained by the above methods is in a free form, the peptide can be converted into an appropriate salt by a publicly known method; when the protein is obtained in a salt form, it can be converted into a free form by a publicly known method.

The polynucleotide encoding the receptor protein of the present invention may be any polynucleotide so long as it contains the base sequence (DNA or RNA, preferably DNA) encoding the receptor protein of the present invention described above. Such a polynucleotide may be a DNA and an RNA including mRNA that encodes the receptor protein of the present invention. The polynucleotide may be either double-stranded or single-stranded. When the polynucleotide is double-stranded, it may be a double-stranded DNA or a DNA:RNA hybrid. When the polynucleotide is single-stranded, it may be a sense strand (i.e., a coding strand) or an antisense strand (i.e., a non-coding strand).

Using the polynucleotide encoding the receptor protein of the present invention, mRNA of the receptor protein of the present invention can be quantified by, for example, the method published in separate volume of Jikken Igaku (Experimental Medical Science), 15 (7), “New PCR and Its Application” (1997) or by a modification of the method.

The DNA encoding the receptor protein of the present invention may be any of genomic DNA, genomic DNA library, cDNA derived from the cells and tissues described above, cDNA library derived from the cells and tissues described above and synthetic DNA. The vector to be used for the library may be any of bacteriophage, plasmid, cosmid and phagemid. The DNA may be directly amplified by reverse transcriptase polymerase chain reaction (hereinafter abbreviated as RT-RCR) using the total RNA or mRNA fraction prepared from the cells and tissues described above.

Specifically, the DNA encoding the receptor protein of the present invention may be any DNA so long as it has, for example, DNA having the amino acid sequence represented by SEQ ID NO: 2 or SEQ ID NO: 3 or it has a base sequence hybridizable to the base sequence represented by SEQ ID NO: 2 or SEQ ID NO: 3 under high stringent conditions and encodes a receptor protein having a substantially equivalent activity (i.e., a ligand binding activity, a signal transduction activity, etc.) as that of the receptor protein of the present invention.

Examples of the DNA that is hybridizable to the base sequence represented by SEQ ID NO: 2 or SEQ ID NO: 3 include a DNA having at least about 90% homology, preferably at least about 95% homology, more preferably at least about 98% homology, much more preferably at least about 99% homology, most preferably at least about 99.5% homology, to the amino acid sequence represented by SEQ ID NO: 2 or SEQ ID NO: 3.

The hybridization can be carried out by publicly known methods or by modifications of these methods, for example, according to the method described in Molecular Cloning, 2nd Ed.; J. Sambrook et al., Cold Spring Harbor Lab. Press, (1989). A commercially available library may also be used according to the instructions of the attached manufacturer's protocol. The hybridization can be carried out preferably under high stringent conditions.

The high stringent conditions used herein are, for example, those in a sodium concentration of about 19 to about 40 mM, preferably about 19 to about 20 mM and a temperature at about 50 to about 70° C., preferably about 60 to about 65° C. In particular, hybridization conditions in a sodium concentration at about 19 mM and a temperature at about 65° C. are most preferred.

More specifically, for the DNA encoding the receptor protein containing the amino acid sequence represented by SEQ ID NO: 1, there may be employed a DNA containing the base sequence represented by SEQ ID NO: 2 or SEQ ID NO: 3, and the like.

The polynucleotide containing a part of the base sequence of the DNA encoding the receptor protein of the present invention or a part of the base sequence complementary to the DNA is intended to include not only the DNA encoding the partial peptide of the present invention described below but also RNA.

According to the present invention, antisense polynucleotides (nucleic acids) that can inhibit replication or expression of a G protein-coupled receptor protein gene can be designed and synthesized based on the base sequence information of the DNA encoding the cloned or determined G protein-coupled receptor protein. Such polynucleotides (nucleic acids) can hybridize to the RNA of the G protein-coupled receptor protein gene and inhibit RNA synthesis or the function of RNA, or can regulate/control the expression of the G protein-coupled receptor protein gene via the interaction with RNAs associated with the G protein-coupled receptor protein. Polynucleotides complementary to the specified sequences of RNA associated with the G protein-coupled receptor protein and polynucleotides that can specifically hybridize to RNA associated with the G protein-coupled receptor protein are useful for regulating and controlling the expression of the G protein-coupled receptor protein gene in vivo and in vitro. These polynucleotides are also useful for the treatment and diagnosis of diseases. The term “correspond” is used to refer to homologous or complementary to a specific sequence of nucleotides, base sequences or nucleic acids including the gene. As between nucleotides, base sequences or nucleic acids and peptides (proteins), the term “corresponding” usually refers to amino acids of a peptide (protein) that is instructed to be derived from the sequence of nucleotides (nucleic acids) or its complements. The 5′ end hairpin loop, 5′ end 6-base-pair repeats, 5′ end untranslated region, polypeptide translation initiation codon, protein coding region, ORF translation initiation codon, 3′ untranslated region, 3′ end palindrome region, and 3′ end hairpin loop of the G protein-coupled receptor protein gene may be selected as preferred target regions, though any region may be a target within the G protein-coupled receptor protein genes.

The relationship between the targeted nucleic acids and the polynucleotides complementary to at least a part of the targeted region, specifically the relationship between the target and the polynucleotides hybridizable to the target, is designated to be “antisense”. The antisense polynucleotides may be polydeoxynucleotides containing 2-deoxy-D-ribose, polydeoxynucleotides containing D-ribose, any other type of polynucleotides which are N-glycosides of purine or pyrimidine bases, or other polymers containing non-nucleotide backbones (e.g., protein nucleic acids and synthetic sequence-specific nucleic acid polymers commercially available) or other polymers containing nonstandard linkages (provided that the polymers contain nucleotides having such a configuration that allows base pairing or base stacking, as is found in DNA and RNA). The antisense polynucleotides may be a double-stranded DNA, a single-stranded DNA, a single-stranded RNA or a DNA:RNA hybrid, and further includes unmodified polynucleotides (or unmodified oligonucleotides), those with publicly known types of modifications, for example, those with labels known in the art, those with caps, methylated polynucleotides, those with substitution of one or more naturally occurring nucleotides with their analogue, those with intramolecular modifications of nucleotides such as those with uncharged linkages (e.g., methyl phosphonates, phosphotriesters, phosphoramidates, carbamates, etc.) and those with charged linkages or sulfur-containing linkages (e.g., phosphorothioates, phosphorodithioates, etc.), those having side chain groups such as proteins (including nucleases, nuclease inhibitors, toxins, antibodies, signal peptides, poly-L-lysine, etc.) or saccharides (e.g., monosaccharides, etc.), those with intercalators (e.g., acridine, psoralen, etc.), those containing chelators (e.g., metals, radioactive metals, boron, oxidative metals, etc.) and those containing alkylating agents, those with modified linkages (e.g., a anomeric nucleic acids, etc.). Herein the terms “nucleoside,” “nucleotide” and “nucleic acid” are used to refer to moieties that contain not only the purine and pyrimidine bases, but may also contain other heterocyclic bases, which have been modified. Such modifications may include methylated purines and pyrimidines, acylated purines and pyrimidines or other heterocyclic rings. Modified nucleotides and modified nucleotides also include modifications on the sugar moiety, for example, wherein one or more hydroxyl groups may optionally be replaced with a halogen, aliphatic groups, etc., or may be converted into the corresponding functional groups such as ethers, amines, or the like.

The antisense polynucleotides (nucleic acids) of the present invention include RNAs, DNAs or modified nucleic acids (RNAs, DNAs). Specific examples of the modified nucleic acids are, but not limited to, sulfurized and thiophosphate derivatives of nucleic acids and those resistant to degradation of polynucleoside or oligonucleoside amides. The antisense nucleic acids of the present invention can be modified preferably based on the following design, that is, by increasing the intracellular stability of the antisense nucleic acids, increasing the cellular permeability of the antisense nucleic acids, increasing the affinity of the nucleic acids to the target sense strand to a higher level, or reducing the toxicity, if any, of the antisense nucleic acids, to a lesser level.

Many such modifications are known in the art, as disclosed in J. Kawakami et al., Pharm. Tech. Japan, Vol. 8, pp. 247, 1992; Vol. 8, pp. 395, 1992; S. T. Crooke et al. ed., Antisense Research and Applications, CRC Press, 1993; etc.

The antisense nucleic acids of the present invention may contain altered or modified sugars, bases or linkages. The antisense nucleic acids may also be provided in a specialized form such as liposomes or microspheres, or may be applied to gene therapy, or may be provided in combination with attached moieties. Such attached moieties include polycations such as polylysine that act as charge neutralizers of the phosphate backbone, or hydrophobic moieties such as lipids (e.g., phospholipids, cholesterols, etc.) that enhance the interaction with cell membranes or increase the uptake of nucleic acids. Preferred examples of the lipids to be attached are cholesterols or derivatives thereof (e.g., cholesteryl chloroformate, cholic acid, etc.). These moieties may be attached to nucleic acids at the 3′ or 5′ ends thereof and may be also attached thereto through a base, sugar, or intramolecular nucleoside linkage. Other moieties may be capping groups specifically placed at the 3′ or 5′ ends of the nucleic acids to prevent degradation by a nuclease such as exonuclease, RNase, etc Such capping groups include, but are not limited to, hydroxyl protecting groups known in the art, including glycols such as polyethylene glycol, tetraethylene glycol, etc.

The inhibitory activity of the antisense nucleic acids can be examined using the transformant of the present invention, the gene expression system of the present invention in vitro or in vivo, or the translation system of the G protein-coupled receptor protein of the present invention in vitro or in vivo. The nucleic acids can be applied to cells by a variety of publicly known methods.

The DNA encoding the partial peptide of the present invention may be any DNA so long as it contains the base sequence encoding the partial peptide of the present invention described above. The DNA may also be any of genomic DNA, genomic DNA library, cDNA derived from the cells and tissues described above, cDNA library derived from the cells and tissues described above and synthetic DNA. The vector to be used for the library may be any of bacteriophage, plasmid, cosmid and phagemid. The DNA may be directly amplified by reverse transcriptase polymerase chain reaction (hereinafter simply referred to as RT-RCR) using mRNA fractions prepared from the cells and tissues described above.

Specific examples of the DNA encoding the partial peptide of the present invention include (1) a DNA that has a part of the base sequence of the DNA containing the base sequence represented by SEQ ID NO: 2 or SEQ ID NO: 3 and (2) a DNA having a base sequence hybridizable to the base sequence represented by SEQ ID NO: 2 or SEQ ID NO: 3 under high stringent conditions and containing a part of the base sequence of the DNA encoding a receptor protein having an activity (i.e., a ligand binding activity, a signal transduction activity, etc.) substantially equivalent to that of the receptor protein peptide of the present invention.

Examples of the DNA that is hybridizable to the base sequence represented by SEQ ID NO: 2 or SEQ ID NO: 3 include a DNA containing the base sequence having at least about 90% homology, preferably at least about 95% homology, more preferably at least about 98% homology, much more preferably at least about 99% homology, most preferably at least about 99.5% homology, to the base sequence represented by SEQ ID NO: 2 or SEQ ID NO: 3, and the like.

For cloning of the DNA that completely encodes the receptor protein or its partial peptide of the present invention (hereinafter sometimes referred to as the receptor protein of the present invention), the DNA may be either amplified by PCR using synthetic DNA primers containing a part of the base sequence of the receptor protein of the present invention, or the DNA inserted into an appropriate vector can be selected by hybridization with a labeled DNA fragment or synthetic DNA that encodes a part or entire region of the receptor protein of the present invention. The hybridization can be carried out, for example, according to the method described in Molecular Cloning, 2nd (J. Sambrook et al., Cold Spring Harbor Lab. Press, 1989), etc. The hybridization may also be performed using a commercially available library in accordance with the protocol described in the instructions attached.

Conversion of the base sequence of DNA can be carried out according to publicly known methods such as the Gupped duplex method, the Kunkel method, etc. or their modifications by using publicly known kits available as Mutan™-super Express Km (Takara Shuzo Co., Ltd.), Mutan™-K (Takara Shuzo Co., Ltd.), etc.

The DNA encoding the cloned receptor protein can be used as it is, depending upon purpose or, if desired, after digestion with a restriction enzyme or after addition of a linker thereto. The DNA may contain ATG as a translation initiation codon at the 5′ end thereof and TAA, TGA or TAG as a translation termination codon at the 3′ end thereof. These translation initiation and termination codons may also be added by using an appropriate synthetic DNA adapter.

The expression vector of the receptor protein of the present invention can be manufactured, for example, by (a) excising the desired DNA fragment from the DNA encoding the receptor protein of the present invention, (b) followed by ligation of the DNA fragment with an appropriate expression vector downstream a promoter in the vector.

Examples of the vector which can be used include plasmids derived form E. coli (e.g., pBR322, pBR325, pUC12, pUC13), plasmids derived from Bacillus subtilis (e.g., pUB110, pTP5, pC194), plasmids derived from yeast (e.g., pSH19, pSH15), bacteriophages such as λ phage, etc., animal viruses such as retrovirus, vaccinia virus, baculovirus, etc. as well as pA1-11, pXT1, pRc/CMV, pRc/RSV, pcDNAI/Neo, etc.

The promoter used in the present invention may be any promoter if it matches well with a host to be used for gene expression. In the case of using animal cells as the host, examples of the promoter include SRα promoter, SV40 promoter, LTR promoter, CMV promoter, HSV-TK promoter, etc.

Among them, CMV promoter or SRα promoter is preferably used Where the host is bacteria of the genus Escherichia, preferred examples of the promoter include trp promoter, lac promoter, recA promoter, λP_{L} promoter, 1 pp promoter, etc. In the case of using bacteria of the genus Bacillus as the host, preferred example of the promoter are SPO1 promoter, SPO2 promoter and penP promoter. In the case of using yeast as the host, preferred examples of the promoter are PHO5 promoter, PGK promoter, GAP promoter and ADH promoter. In the case of using insect cells as the host, preferred examples of the promoter include polyhedrin promoter, P10 promoter, and the like.

In addition to the foregoing examples, the expression vector may further optionally contain an enhancer, a splicing signal, a poly A addition signal, a selection marker, SV40 replication origin (hereinafter sometimes abbreviated as SV40ori) etc. Examples of the selection marker include dihydrofolate reductase (hereinafter sometimes abbreviated as dhfr) gene [methotrexate (MTX) resistance], ampicillin resistant gene (hereinafter sometimes abbreviated as Ampr), neomycin resistant gene (hereinafter sometimes abbreviated as Neo, G418 resistance), etc. In particular, when CHO (dhfr⁻) cell is used together with dhfr gene as the selection marker, selection can also be made by using a thymidine free medium.

If necessary, a signal sequence that matches with a host is added to the N-terminus of the receptor protein of the present invention. Examples of the signal sequence that can be used are Pho A signal sequence, OmpA signal sequence, etc. in the case of using bacteria of the genus Escherichia as the host; α-amylase signal sequence, subtilisin signal sequence, etc. in the case of using bacteria of the genus Bacillus as the host; MFα signal sequence, SUC2 signal sequence, etc. in the case of using yeast as the host; and insulin signal sequence, α-interferon signal sequence, antibody molecule signal sequence, etc. in the case of using animal cells as the host, respectively.

Using the vector containing the DNA encoding the receptor protein of the present invention thus constructed, transformants can be manufactured.

Examples of the host, which may be employed, are bacteria belonging to the genus Escherichia, bacteria belonging to the genus Bacillus, yeast, insect cells, insects and animal cells, etc.

Specific examples of bacteria belonging to the genus Escherichia include Escherichia coli K12 DHi [Proc. Natl. Acad. Sci. U.S.A., 60, 160 (1968)), JM103 (Nucleic Acids Research, 9, 309 (1981)), JA221 (Journal of Molecular Biology, 120, 517 (1978)], HB101 [Journal of Molecular Biology, 41, 459 (1969)), C600 (Genetics, 39, 440 (1954)], etc.

Examples of bacteria belonging to the genus Bacillus include Bacillus subtilis MI114 [Gene, 24, 255 (1983)], 207-21 [Journal of Biochemistry, 95, 87 (1984)], etc.

Examples of yeast include Saccharoinyces cereviseae AH22, AH22R⁻, NA87-11A, DKD-5D, 20B-12, Schizosaccharomyces pombe NCYC1913, NCYC2036, Pichia pastoris, etc.

Examples of insect cells include, for the virus AcNPV, Spodoptera frugiperda cell (Sf cell), MGI cell derived from mid-intestine of Trichoplusia ni, High Five™ cell derived from egg of Trichoplusia ni, cells derived from Mamestra brassicae, cells derived from Estigmenza acrea, etc.; and for the virus BmNPV, Bombyx mori N cell (BmN cell), etc. Examples of the Sf cell which can be used are Sf9 cell (ATCC CRL1711), Sf21 cell (both cells are described in Vaughn, J. L. et al., In Vivo, 13, 213-217 (1977), etc.

As the insect, for example, a larva of Bombyx mori can be used [Maeda et al., Nature, 315, 592 (1985)].

Examples of animal cells include monkey cell COS-7, Vero, Chinese hamster cell CHO (hereinafter referred to as CHO cell), dhfr gene deficient Chinese hamster cell CHO (hereinafter simply referred to as CHO(dhfr⁻) cell), mouse L cell, mouse AtT-20, mouse myeloma cell, rat GH 3, human FL cell, etc.

Bacteria belonging to the genus Escherichia can be transformed, for example, according to the method described in Proc. Natl. Acad. Sci. U.S.A., 69, 2110 (1972) or Gene, 17, 107 (1982), etc.

Bacteria belonging to the genus Bacillus can be transformed, for example, according to the method described in Molecular & General Genetics, 168, 111 (1979), etc.

Yeast can be transformed, for example, according to the method described in Methods in Enzymology, 194, 182-187 (1991) or Proc. Natl. Acad. Sci. U.S.A., 75, 1929 (1978), etc.

Insect cells or insects can be transformed, for example, according to the method described in Bio/Technology, 6, 47-55(1988), etc.

Animal cells can be transformed, for example, according to the method described in Saibo Kogaku (Cell Engineering), extra issue 8, Shin Saibo Kogaku Jikken Protocol (New Cell Engineering Experimental Protocol), 263-267 (1995), published by Shujunsha, or Virology, 52, 456 (1973).

Thus, the transformant transformed with the expression vector containing the DNA encoding the G protein-coupled receptor protein can be obtained.

Where the host is bacteria belonging to the genus Escherichia or the genus Bacillus, the transformant can be appropriately incubated in a liquid medium which contains materials required for growth of the transformant such as carbon sources, nitrogen sources, inorganic materials, etc. Examples of the carbon sources include glucose, dextrin, soluble starch, sucrose, etc. Examples of the nitrogen sources include inorganic or organic materials such as ammonium salts, nitrate salts, corn steep liquor, peptone, casein, meat extract, soybean cake, potato extract, etc. Examples of the inorganic materials are calcium chloride, sodium dihydrogenphosphate, magnesium chloride, etc. In addition, yeast, vitamins, growth promoting factors etc. may also be added to the medium. Preferably, pH of the medium is adjusted to about 5 to about 8.

A preferred example of the medium for incubation of the bacteria belonging to the genus Escherichia is M9 medium supplemented with glucose and Casamino acids (Miller, Journal of Experiments in Molecular Genetics, 431-433, Cold Spring Harbor Laboratory, New York, 1972). If necessary, a chemical such as 3β-indolylacrylic acid can be added to the medium thereby to activate the promoter efficiently.

Where the bacteria belonging to the genus Escherichia are used as the host, the transformant is usually cultivated at about 15 to about 43° C. for about 3 hours to about 24 hours. If necessary, the culture may be aerated or agitated.

Where the bacteria belonging to the genus Bacillus are used as the host, the transformant is cultivated generally at about 30 to about 40° C. for about 6 hours to about 24 hours. If necessary, the culture can be aerated or agitated.

Where yeast is used as the host, the transformant is cultivated in, for example, Burkholder's minimal medium [Bostian, K. L. et al., Proc. Natl. Acad. Sci. U.S.A., 77, 4505 (1980)] or in SD medium supplemented with 0.5% Casamino acids [Bitter, G. A. et al., Proc. Natl. Acad. Sci. U.S.A., 81, 5330 (1984)]. Preferably, pH of the medium is adjusted to about 5 to about 8. In general, the transformant is cultivated at about 20° C. to about 35° C. for about 24 hours to about 72 hours. If necessary, the culture can be aerated or agitated.

Where insect cells or insects are used as the host, the transformant is cultivated in, for example, Grace's Insect Medium [Grace, T. C. C., Nature, 195, 788 (1962)] to which an appropriate additive such as immobilized 10% bovine serum is added. Preferably, pH of the medium is adjusted to about 6.2 to about 6.4. Normally, the transformant is cultivated at about 27° C. for about 3 days to about 5 days and, if necessary, the culture can be aerated or agitated.

Where animal cells are employed as the host, the transformant is cultivated in, for example, MEM medium containing about 5% to about 20% fetal bovine serum (Science, 122, 501 (1952)], DMEM medium [Virology, 8, 396 (1959)), RPMI 1640 medium [The Journal of the American Medical Association, 199, 519 (1967)], 199 medium [Proceeding of the Society for the Biological Medicine, 73, 1 (1950)], etc. Preferably, pH of the medium is adjusted to about 6 to about 8. The transformant is usually cultivated at about 30° C. to about 40° C. for about 15 hours to about 60 hours and, if necessary, the culture can be aerated or agitated.

As described above, the G protein-coupled receptor protein of the present invention can be produced in the cells or cell membranes of the transformants, or outside the transformants.

The receptor protein of the present invention can be separated and purified from the culture described above by the following procedures.

When the receptor protein of the present invention is extracted from the culture or cells, after cultivation, the transformants or cells is collected by publicly known methods and suspended in an appropriate buffer. The transformants or cells are then disrupted by publicly known methods such as ultrasonication, a treatment with lysozyme and/or freeze-thaw cycling, etc., followed by centrifugation, filtration, etc. Thus, the crude extract of the receptor protein can be obtained. The buffer used for the procedures may contain a protein modifier such as urea or guanidine hydrochloride, or a surfactant such as Triton X-100™, etc. When the receptor protein is secreted in the culture broth, after completion of the cultivation the supernatant can be separated from the transformants or cells to collect the supernatant by publicly known methods.

The receptor protein contained in the supernatant or extract thus obtained can be purified by appropriately combining the publicly known methods for separation and purification. Such publicly known methods for separation and purification include a method utilizing difference in solubility such as salting out, solvent precipitation, etc.; a method mainly utilizing difference in molecular weight such as dialysis, ultrafiltration, gel filtration, SDS-polyacrylamide gel electrophoresis, etc.; a method utilizing difference in electric charge such as ion exchange chromatography, etc.; a method utilizing difference in specific affinity such as affinity chromatography, etc.; a method utilizing difference in hydrophobicity such as reverse phase high performance liquid chromatography, etc.; a method utilizing difference in isoelectric point such as isoelectrofocusing electrophoresis; and the like.

When the receptor protein thus obtained is in a free form, it can be converted into the salt by publicly known methods or modifications thereof. On the other hand, when the receptor protein is obtained in the form of a salt, it can be converted into the free form or in the form of a different salt by publicly known methods or modifications thereof.

The receptor protein produced by the recombinant can be treated, prior to or after the purification, with an appropriate protein-modifying enzyme so that the receptor protein can be appropriately modified to partially remove a polypeptide. Examples of the protein-modifying enzyme include trypsin, chymotrypsin, arginyl endopeptidase, protein kinase, glycosidase and the like.

The activity of the thus produced receptor protein of the present invention or salts thereof can be determined by a binding test to a labeled ligand, by an enzyme immunoassay using a specific antibody, or the like.

The antibody to the receptor protein of the present invention or its peptide, or salts thereof may be any of polyclonal and monoclonal antibodies, so long as they can recognize the receptor protein of the present invention or its peptide, or salts thereof.

The antibody to the receptor protein of the present invention or its peptide, or salts thereof (hereinafter sometimes merely referred to as the receptor protein or the like of the present invention) can be manufactured by publicly known methods for manufacturing antibodies or antisera, using as an antigen the receptor protein or the like of the present invention.

[Preparation of Monoclonal Antibody]

(a) Preparation of Monoclonal Antibody-Producing Cells

The receptor protein or the like of the present invention is administered to a mammal, either solely or together with carriers or diluents to the site that can produce the antibody by the administration. In order to potentiate the antibody productivity upon the administration, complete Freund's adjuvant or incomplete Freund's adjuvant may be administered. The administration is effected usually once every 2 to 6 weeks and approximately 2 to 10 times in total. The mammals to be used include monkey, rabbit, dog, guinea pig, mouse, rat, sheep and goat, with mouse and rat being preferred.

In the preparation of the monoclonal antibody-producing cells, an animal wherein the antibody titer is noted is selected from warm-blooded animals immunized with antigens, e.g., mice, then spleen or lymph node is collected after two to five days from the final immunization and the antibody-producing cells contained therein are fused with myeloma cells to give monoclonal antibody-producing hybridomas. The antibody titer in antisera may be determined, for example, by reacting a labeled form of the receptor protein or the like of the present invention, which will be described later, with the antiserum followed by measuring the binding activity of the labeling agent bound to the antibody. The fusion may be carried out, for example, according to the method of Koehler and Milstein (Nature, 256, 495, 1975). Examples of the fusion accelerator are polyethylene glycol (PEG), Sendai virus, etc. and PEG is preferably used.

Examples of myeloma cells include NS-1, P3U1, SP2/0, etc., with P3U1 being preferred. The ratio of the number of the antibody-producing cells (spleen cells) to the number of myeloma cells to be used is preferably about 1:1 to about 20:1 and PEG (preferably PEG 1000 to PEG 6000) is added in a concentration of about 10% to about 80%. The cell fusion can be efficiently carried out by incubating both cells at about 20° C. to about 40° C., preferably about 30° C. to about 37° C. for about 1 minute to about 10 minutes.

Various methods can be used for screening of a monoclonal antibody-producing hybridoma. Examples of such methods include a method which comprises adding the supernatant of hybridoma to a solid phase (e.g., microplate) adsorbed with the receptor protein or the like as antigen directly or together with a carrier, adding an anti-immunoglobulin antibody (where mouse cells are used for the cell fusion, anti-mouse immunoglobulin antibody is used) labeled with a radioactive substance or an enzyme or Protein A and detecting the monoclonal antibody bound to the solid phase, and a method which comprises adding the supernatant of hybridoma to a solid phase adsorbed with an anti-immunoglobulin antibody or Protein A, adding the receptor protein or the like labeled with a radioactive substance or an enzyme and detecting the monoclonal antibody bound to the solid phase.

The monoclonal antibody can be selected according to publicly known methods or their modifications. In general, the selection can be effected in a medium for animal cells supplemented with HAT (hypoxanthine, aminopterin and thymidine). Any selection and growth medium can be employed as far as the hybridoma can grow therein. For example, RPMI 1640 medium containing 1% to 20%, preferably 10% to 20% fetal bovine serum, GIT medium (Wako Pure Chemical Industries, Ltd.) containing 1% to 10% fetal bovine serum, a serum free medium for cultivation of a hybridoma (SFM-101, Nissui Seiyaku Co., Ltd.) and the like can be used for the selection and growth medium. The cultivation is carried out generally at 20° C. to 40° C., preferably 37° C., for about 5 days to about 3 weeks, preferably 1 to 2 weeks, normally in 5% CO₂. The antibody titer of the culture supernatant of a hybridoma can be determined as in the determination of antibody titer in antisera described above.

(b) Purification of Monoclonal Antibody

Separation and purification of a monoclonal antibody can be carried out according the same manner as applied to conventional separation and purification for polyclonal antibodies, such as separation and purification of immunoglobulins [for example, salting-out, alcohol precipitation, isoelectric point precipitation, electrophoresis, adsorption and desorption with ion exchangers (e.g., DEAE), ultracentrifugation, gel filtration, or a specific purification method which comprises collecting only an antibody with an activated adsorbent such as an antigen-binding solid phase, Protein A or Protein G and dissociating the binding to obtain the antibody].

[Preparation of Polyclonal Antibody]

The polyclonal antibody of the present invention can be manufactured by publicly known methods or modifications thereof. For example, a complex of immunogen (an antigen of the receptor protein or the like) and a carrier protein is formed and a mammal is immunized with the complex in a manner similar to the method described above for the manufacture of monoclonal antibody. The product containing the antibody to the receptor protein or the like of the present invention is collected from the immunized animal followed by separation and purification of the antibody.

In the complex of immunogen and carrier protein for immunizing mammals, the type of carrier protein and the mixing ratio of carrier to hapten may be any type and in any ratio, as long as the antibody is efficiently produced to the hapten immunized by crosslinking to the carrier. For example, bovine serum albumin, bovine thyroglobulin or keyhole limpet hemocyanin is coupled to hapten in a carrier-to-hapten weight ratio of approximately 0.1 to 20, preferably 1 to 5.

A variety of condensation agents can be used for the coupling of carrier to hapten. Glutaraldehyde, carbodiimide, maleimide activated ester and activated ester reagents containing thiol group or dithiopyridyl group are used for the coupling.

The condensation product is administered to warm-blooded animals either solely or together with carriers or diluents to the site that can produce the antibody by the administration. In order to potentiate the antibody productivity upon the administration, complete Freund's adjuvant or incomplete Freund's adjuvant may be administered. The administration is usually carried out once approximately every 2 to 6 weeks and about 3 to about 10 times in total.

The polyclonal antibody can be collected from the blood, ascites, etc., preferably from the blood of mammals immunized by the method described above.

The polyclonal antibody titer in antiserum can be determined by the same procedure as in the serum antibody titer described above. The polyclonal antibody can be separated and purified according to the same method for separation and purification of immunoglobulin as used for the monoclonal antibody described above.

The receptor protein of the present invention or salts thereof, its partial peptide or salts thereof and the DNA encoding the receptor protein or its partial peptide can be used: (1) for the determination of a ligand (an agonist) to the G protein-coupled receptor protein of the present invention, (2) as the agent for the prevention and/or treatment of disease associated with dysfunction of the G protein-coupled receptor protein of the present invention, (3) as the genetic diagnostic agent, (4) for the screening of a compound that alters the expression level of the receptor protein of the present invention or its partial peptide, (5) as the agent for the prevention and/or treatment of various diseases, comprising a compound that alters the expression level of the receptor protein of the present invention or its partial peptide, (6) for the quantification of a ligand to the G protein-coupled receptor protein of the present invention, (7) for the screening of a compound (an agonist, an antagonist, etc.) that alters the binding property between the G protein-coupled receptor protein of the present invention and a ligand, (8) as the agent for the prevention and/or treatment of various diseases, comprising a compound (agonist, antagonist, etc.) that alters the binding property between the G protein-coupled receptor protein of the present invention and a ligand, (9) for the quantification of the receptor protein of the present invention or its partial peptide, or salts thereof, (10) for the screening of a compound that alters the amount of the receptor protein of the present invention or its partial peptide on a cell membrane, (11) as the agent for the prevention and/or treatment of various diseases, comprising a compound that alters the amount of the receptor protein of the present invention or its partial peptide on a cell membrane, (12) for the neutralization by the antibody to the receptor protein of the present invention or its partial peptide, or salts thereof, (13) for the preparation of non-human animal bearing the DNA encoding the G protein-coupled receptor protein of the present invention, and the like.

In particular, the compound (e.g., an agonist, an antagonist, etc.) that alters the binding property of a ligand to a G protein-coupled receptor specific to human or other mammals can be screened, using the receptor binding assay system by applying the expression system of the recombinant G protein-coupled receptor protein of the present invention, and agonist or antagonist can be used as a prophylactic/therapeutic agent for various diseases.

Hereinafter, the receptor protein of the present invention or its partial peptide, or salts thereof (hereinafter sometimes merely referred to as the receptor protein or the like of the present invention), the DNA encoding the receptor protein of the present invention or its partial peptide (hereinafter sometimes collectively referred to as the DNA of the present invention) and the antibodies to the receptor protein or the like of the present invention (hereinafter sometimes referred to as the antibody of the present invention) are specifically described with reference to their use.

(1) Determination of a Ligand (an Agonist) to the G Protein-Coupled Receptor Protein of the Present Invention

The receptor protein of the present invention or its salt or the partial peptide of the present invention or its salt is useful as a reagent for searching and determining a ligand (an agonist) to the receptor protein of the present invention or salts thereof.

That is, the present invention provides a method of determining a ligand to the receptor protein of the present invention, which comprises contacting the receptor protein of the present invention or salts thereof or the partial peptide of the present invention or salts thereof with a test compound.

Examples of the compounds to be tested include publicly known ligands (e.g., angiotensin, bombesin, canavinoid, cholecystokinin, glutamine, serotonin, melatonin, neuropeptide Y, opioid, purines, vasopressin, oxytocin, PACAP, secretin, glucagon, calcitonin, adrenomedulin, somatostatin, GHRH, CRF, ACTH, GRP, PTH, VIP (vasoactive intestinal and related polypeptide), somatostatin, dopamine, motilin, amylin, bradykinin, CGRP (calcitonin gene related peptide), leukotrienes, pancreastatin, prostaglandins, thromboxane, adenosine, adrenaline, α and β-chemokines (e.g., IL-8, GROα, GROβ, GROγ, NAP-2, ENA-78, PF4, IP10, GCP-2, MCP-1, HC14, MCP-3, I-309, MIP1α, MIP-1β, RANTES, etc.), endothelin, enterogastrin, histamine, neurotensin, TRH, pancreatic polypeptide or galanin, lysophosphatidic acid (LPA), sphingosine-1-phosphate, etc.) as well as, for example, tissue extracts and cell culture supernatants from humans or mammals (e.g., mice, rats, swine, bovine, sheep, monkeys, etc.). For example, the tissue extract, cell culture supernatant or the like is added to the receptor protein of the present invention and fractionated while assaying the cell-stimulating activities, whereby a single ligand can be finally obtained.

Specifically, the method of determining a ligand of the present invention comprises either using the receptor protein of the present invention, its partial peptide or salts thereof, or using the constructed recombinant receptor protein expression system in the receptor binding assay, thereby to determine a compound (e.g., a peptide, a protein, a non-peptide compound, a synthetic compound, a fermentation product, etc.) or its salts that bind to the receptor protein of the present invention to provide a cell stimulating activity (e.g., activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.).

The method of determining a ligand of the present invention is characterized, for example, by measuring the amount of a test compound bound to the receptor protein or its partial peptide of the present invention or assaying the cell stimulating activity, etc., when the receptor protein or its partial peptide of the present invention is brought in contact with the test compound.

More specifically, the present invention provides:

(1) A method of determining a ligand to the receptor protein of the present invention or a salt thereof, which comprises contacting a labeled test compound with the receptor protein of the present invention or its salt or the partial peptide of the present invention or its salt and measuring the amount of the labeled test compound bound to the protein or its salt or to the partial peptide or its salt;

(2) A method of determining a ligand to the receptor protein of the present invention or a salt thereof, which comprises contacting a labeled test compound with a cell containing the receptor protein of the present invention or with a membrane fraction of the cell and measuring the amount of the labeled test compound bound to the cell or the membrane fraction;

(3) A method of determining a ligand to the receptor protein of the present invention, which comprises culturing a transformant containing a DNA encoding the receptor protein of the present invention, contacting a labeled test compound with the receptor protein expressed on a cell membrane by said culturing, and measuring the amount of the labeled test compound bound to the expressed receptor protein or a salt thereof;

(4) A method of determining a ligand to the receptor protein of the present invention or a salt thereof, which comprises assaying the receptor protein-mediated cell stimulating activity (e.g., the activity that promotes or suppresses arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.), when a test compound is brought in contact with a cell containing the receptor protein of the present invention; and,

(5) A method of determining a ligand to the receptor protein of the present invention or a salt thereof, which comprises assaying the receptor protein-mediated cell stimulating activity (e.g., the activity that promotes or suppresses arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.), when a test compound is brought in contact with the receptor protein expressed on a cell membrane by culturing a transformant containing a DNA encoding the receptor protein of the present invention.

In particular, it is preferred to perform the methods (1) to (3) described above to confirm that a test compound can bind to the receptor protein of the present invention, followed by the methods (4) and (5) described above.

Any protein can be used for the method of the present invention for determining ligands, so long as it contains the receptor protein of the present invention or the partial peptide of the present invention. However, the receptor proteins abundantly expressed using animal cells are appropriate for the present invention.

The receptor protein of the present invention can be manufactured by the expression methods described above, preferably by expressing a DNA encoding the receptor protein in mammalian or insect cells. DNA fragments encoding the desired portion of the protein include, but are not limited to, complementary DNA. For example, gene fragments or synthetic DNA may be used as well. For introducing a DNA fragment encoding the receptor protein of the present invention into host animal cells and efficiently expressing the same, it is preferred to insert the DNA fragment downstream the polyhedrin promoter of nuclear polyhedrosis virus (NPV), which is a baculovirus having insect hosts, an SV40-derived promoter, a retrovirus promoter, a metallothionein promoter, a human heat shock promoter, a cytomegalovirus promoter, an SRα promoter or the like. The quantity and quality of the receptor expressed can be determined by a publicly known method. For example, this determination can be made by the method described in the literature [Nambi, P. et al., J. Biol. Chem., Vol. 267, pp. 19555-19559 (1992)].

Accordingly, the substance containing the receptor protein of the present invention, its partial peptide or a salt thereof in the methods of determining ligands may be a receptor protein purified by publicly known method, or its partial peptide or salts thereof, or may be a cell containing the receptor protein or a membrane fraction of such a cell.

Where cells containing the receptor protein of the present invention are used for the methods of the present invention for determination of ligands, the cells may be fixed with glutaraldehyde, formalin, etc. The fixation can be made by publicly known methods.

The cells containing the receptor protein of the present invention refer to host cells that have expressed the receptor protein of the present invention, which host cells include Escherichia coli, Bacillus subtilis, yeast, insect cells, animal cells, etc.

The cell membrane fractions refer to fractions abundant in cell membranes obtained by cell disruption and subsequent fractionation by publicly known methods. Useful cell disruption methods include cell squashing using a Potter-Elvehjem homogenizer, disruption using a Waring blender or Polytron (manufactured by Kinematica Inc.), disruption by ultrasonication, and disruption by cell spraying via a thin nozzle under increased pressure using a French press or the like. Cell membrane fractionation is effected mainly by fractionation using a centrifugal force, such as centrifugation for fractionation and density gradient centrifugation. For example, cell disruption fluid is centrifuged at a low rate (500 to 3,000 rpm) for a short period of time (normally about 1 to 10 minutes), the resulting supernatant is then centrifuged at a higher rate (15,000 to 30,000 rpm) normally for 30 minutes to 2 hours. The precipitate thus obtained is used as the membrane fraction. The membrane fraction is rich in the receptor protein expressed and membrane components such as cell-derived phospholipids, membrane proteins, etc.

The amount of the receptor protein in the cells containing the receptor protein or in the membrane fraction is preferably 10³ to 10⁸ molecules per cell, more preferably 10⁵ to 10⁷ molecules per cell. As the amount of expression increases, the ligand binding activity per unit of membrane fraction (specific activity) increases so that not only the highly sensitive screening system can be constructed but also large quantities of samples can be assayed with the same lot.

To perform the methods (1) through (3) for determination of a ligand to the receptor protein of the present invention or salts thereof, an appropriate receptor protein fraction and a labeled test compound are required.

The receptor protein fraction is preferably a fraction of naturally occurring receptor protein or a recombinant receptor fraction having an activity equivalent to that of the natural receptor protein. Herein, the equivalent activity is intended to mean a ligand binding activity, a signal transduction activity or the like that is equivalent to that possessed by naturally occurring receptor proteins.

Preferred examples of the labeled test compounds include [³H]—, [¹²⁵I]—, [¹⁴C]—, [³⁵S]—, etc. labeled angiotensin, bombesin, canavinoid, cholecystokinin, glutamine, serotonin, melatonin, neuropeptide Y, opioid, purines, vasopressin, oxytocin, PACAP, secretin, glucagon, calcitonin, adrenomedulin, somatostatin, GHRH, CRF, ACTH, GRP, PTH, VIP (vasoactive intestinal and related polypeptide), somatostatin, dopamine, motilin, amylin, bradykinin, CGRP (calcitonin gene related peptide), leukotrienes, pancreastatin, prostaglandins, thromboxane, adenosine, adrenaline, α and β-chemokines (e.g., IL-8, GROα, GROβ, GROγ, NAP-2, ENA-78, PF4, IP10, GCP-2, MCP-1, HC14, MCP-3, I-309, MIP-1α, MIP-1β, RANTES, etc.), endothelin, enterogastrin, histamine, neurotensin, TRH, pancreatic polypeptide, galanin, lysophosphatidic acid (LPA), sphingosine-1-phosphate, etc.

Specifically, the ligand to the receptor protein of the present invention or a salt thereof is determined by the following procedures. First, a receptor preparation is prepared by suspending cells containing the receptor protein of the present invention or the membrane fraction of the cells in a buffer solution appropriate for use in the determination methods. Any buffer may be used so long as it does not interfere with ligand-receptor binding, such buffers including a phosphate buffer or a Tris-HCl buffer having pH of 4 to 10 (preferably pH of 6 to 8). For the purpose of minimizing non-specific binding, a surfactant such as CHAPS, Tween-80™ (manufactured by Kao-Atlas Inc.), digitonin, deoxycholate, etc. and various proteins such as bovine serum albumin or gelatin, may optionally be added to the buffer. Furthermore, for the purpose of preventing the degradation of receptors or ligands by a protease, protease inhibitors such as PMSF, leupeptin, E-64 (manufactured by Peptide Institute, Inc.), pepstatin etc. may also be added. A given amount (5,000 to 500,000 cpm) of a test compound labeled with [³H], [¹²⁵I], [¹⁴C], [³⁵S], etc. is added to 0.01 ml to 10 ml of the receptor solution. To determine the amount of non-specific binding (NSB), a reaction tube containing an unlabeled test compound in a large excess is also provided. The reaction is carried out at approximately 0° C. to 50° C., preferably about 4° C. to 37° C. for about 20 minutes to about 24 hours, preferably about 30 minutes to 3 hours. After completion of the reaction, the reaction mixture is filtrated through glass fiber filter paper, etc. and washed with an appropriate amount of the same buffer. The residual radioactivity in the glass fiber filter paper is then measured by means of a liquid scintillation counter or y-counter.

A test compound exceeding 0 cpm in count obtained by subtracting nonspecific binding (NSB) from the total binding (B) (B minus NSB) may be selected as a ligand (an agonist) to the receptor protein of the present invention or salts thereof.

The method (4) or (5) above for determination of a ligand to the receptor protein of the present invention or salts thereof can be performed as follows. The receptor protein-mediated cell-stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.) may be determined by publicly known methods, or using assay kits commercially available. Specifically, the cells containing the receptor protein are first cultured on a multiwell plate, etc. Prior to the ligand determination, the medium is replaced with fresh medium or with an appropriate non-cytotoxic buffer, followed by incubation for a given period of time in the presence of a test compound, etc. Subsequently, the cells are extracted or the supernatant is recovered and the resulting product is quantified by appropriate procedures. Where it is difficult to detect the production of the indicator substance for the cell-stimulating activity (e.g., arachidonic acid, etc.) due to a degrading enzyme contained in the cells, an inhibitor against such a degradation enzyme may be added prior to the assay. For detecting the activities such as the cAMP production suppression activity, etc., the baseline production in the cells is increased by forskolin or the like and the suppressing effect on the increased baseline production can then be detected.

The kit of the present invention for determination of a ligand that binds to the receptor protein of the present invention or salts thereof comprises the receptor protein of the present invention or salts thereof, the partial peptide of the present invention or salts thereof, the cells containing the receptor protein of the present invention, the membrane fraction of the cells containing the receptor protein of the present invention, etc.

Examples of the ligand determination kit of the present invention are given below.

1. Reagents for Determining Ligands

(1) Assay and Wash Buffers

Hanks' Balanced Salt Solution (manufactured by Gibco Co.) supplemented with 0.05% bovine serum albumin (Sigma Co.).

The solution is sterilized by filtration through a 0.45 μm filter and stored at 4° C. Alternatively, the solution may be prepared at use.

(2) G Protein-Coupled Receptor Protein Preparation

CHO cells on which the receptor protein of the present invention has been expressed are subcultured in a 12-well plate at the rate of 5×10⁵ cells/well and then cultured at 37° C. under 5% CO₂ and 95% air for 2 days.

(3) Labeled Test Compound

A compound labeled with commercially available [³H], [¹²⁵I], [¹⁴C], [³⁵S], etc., or a compound labeled by appropriate methods.

An aqueous solution of the compound is stored at 4° C. or −20° C. The solution is diluted to 1 μM with an assay buffer at use. A sparingly water-soluble test compound is dissolved in dimethylformamide, DMSO, methanol, or the like.

(4) Unlabeled Compound

An unlabeled form of the same compound as the labeled compound is prepared in a concentration 100 to 1,000-fold higher than that of the labeled compound.

2. Assay Method

(1) CHO cells expressing the receptor protein of the present invention are cultured in a 12-well culture plate. After washing twice with 1 ml of an assay buffer, 490 μl of the assay buffer is added to each well.

(2) After 5 μl of the labeled test compound is added, the resulting mixture is incubated at room temperature for an hour. To determine the non-specific binding, 5 μl of the unlabeled compound is added to the system.

(3) The reaction mixture is removed and the wells are washed 3 times with 1 ml of wash buffer. The labeled test compound bound to the cells is dissolved in 0.2N NaOH-1% SDS and then mixed with 4 ml of liquid scintillator A (manufactured by Wako Pure Chemical Industries, Ltd.).

(4) The radioactivity is measured using a liquid scintillation counter (manufactured by Beckman Co.).

The ligands that can bind to the receptor protein of the present invention or salts thereof include substances present specifically in the brain, pituitary gland, pancreas, etc. Examples of such ligands are angiotensin, bombesin, canavinoid, cholecystokinin, glutamine, serotonin, melatonin, neuropeptide Y, opioids, purines, vasopressin, oxytocin, PACAP, secretin, glucagon, calcitnonin, adrenomedulin, somatostatin, GHRH, CRF, ACTH, GRP, PTH, VIP (vasoactive intestinal and related polypeptide), somatostatin, dopamine, motilin, amylin, bradykinin, CGRP (calcitonin gene related peptide), leukotrienes, pancreastatin, prostaglandins, thromboxane, adenosine, adrenaline, a and P-chemokines (e.g., IL-8, GROα, GROβ, GROγ, NAP-2, ENA-78, PF4, IP10, GCP-2, MCP-1, HC14, MCP-3, I-309, MIP-1α, MIP-1β, RANTES, etc.), endothelin, enterogastrin, histamine, neurotensin, TRH, pancreatic polypeptide, galanin, lysophosphatidic acid (LPA), sphingosine-1-phosphate, etc.

(2) Prophylactic and/or Therapeutic Agents for Diseases Associated with the Dysfunction of the G Protein-Coupled Receptor Protein of the Present Invention

When a compound is clarified to be a ligand to the receptor protein of the present invention by the methods described in (1) above, (i) the receptor protein of the present invention, or (ii) the DNA encoding the receptor protein can be used, depending on the activities possessed by the ligand, as a prophylactic and/or therapeutic agent for diseases associated with dysfunction of the receptor protein of the present invention.

For example, when any physiological activity of the receptor protein of the present invention cannot be expected due to a reduced level of the receptor protein in a patient (deficiency of the receptor protein), the ligand activity can be exhibited by the following methods: (1) the receptor protein of the present invention is administered to the patient to supplement the amount of the receptor protein; or (2) the amount of the receptor protein of the present invention is increased in the patient by: a) administration of the DNA encoding the receptor protein of the present invention to the patient for expression, or by b) insertion of the DNA encoding the receptor protein of the present invention in the target cells for expression, and the cells thus expressed are then transplanted to the patient. Thus, the amount of the receptor protein can be increased in the patient, whereby the ligand activity can be exhibited sufficiently. Therefore, the DNA encoding the receptor protein of the present invention is useful as a safe and low toxic prophylactic and/or therapeutic drug for diseases associated with the dysfunction of the receptor protein of the present invention.

The receptor protein of the present invention is found to have about 44% homology, about 42% homology, about 40% homology and about 86% homology, on an amino acid sequence level, respectively, to human EDG-1 receptor, human EDG-5 receptor, human EDG-3 receptor and rat nrg-1 (NGF-repressed G protein-coupled receptor protein: Molecular and Cellular Neuroscience, 14, 141-152 (1999)), which are all G protein-coupled receptor proteins.

The receptor protein of the present invention is useful for the prevention and/or treatment of central dysfunction (e.g., Alzheimer's disease, senile dementia, eating disorder, etc.), inflammatory diseases (e.g., allergy, asthma, rheumatoid, etc.), circulatory diseases (e.g., hypertension, cardiac hypertrophy, angina pectoris, arteriosclerosis, etc.), cancer (e.g., non-small cell lung cancer, ovarian cancer, prostate cancer, gastric cancer, bladder cancer, breast cancer, cervical cancer, colon cancer, rectal cancer, etc.), diabetes mellitus, etc.

When the receptor protein of the present invention is used as the prophylactic/therapeutic agents supra, the receptor protein can be prepared into pharmaceutical compositions in a conventional manner.

On the other hand, when the DNA encoding the receptor protein of the present invention (hereinafter sometimes referred to as the DNA of the present invention) is used as a prophylactic/therapeutic agent described above, the DNA of the present invention may be administered alone; alternatively, the DNA is inserted into an appropriate vector such as retrovirus vector, adenovirus vector, adenovirus-associated virus vector, etc. and then administered in a conventional manner. The DNA of the present invention can also be administered as naked DNA, or with adjuvants to assist its uptake by gene gun or through a catheter such as a catheter with a hydrogel.

For example, (1) the receptor protein of the present invention, or (2) the DNA encoding the receptor protein can be used orally in the form of tablets which, if necessary, may be sugar coated, capsules, elixir, microcapsules, etc., or parenterally in the form of injectable preparations such as a sterile solution, a suspension, etc. in water or with other pharmaceutically acceptable liquid. For example, these preparations can be manufactured by mixing (1) the receptor protein of the present invention or (2) the DNA encoding the receptor protein with a physiologically acceptable known carrier, a flavoring agent, an excipient, a vehicle, an antiseptic, a stabilizer, a binder, etc. in a unit dosage form required in a generally accepted manner applied to making pharmaceutical preparations. The active ingredient in the preparation is controlled in such a dose that an appropriate dose is obtained within the specified range given.

Additives miscible with tablets, capsules etc. include a binder such as gelatin, corn starch, tragacanth and gum arabic, an excipient such as crystalline cellulose, a swelling agent such as corn starch, gelatin, alginic acid, etc., a lubricant such as magnesium stearate, a sweetening agent such as sucrose, lactose and saccharin, and a flavoring agent such as peppermint, akamono oil and cherry. When the unit dosage is in the form of capsules, liquid carriers such as oils and fats may further be used together with the additives described above. A sterile composition for injection may be formulated according to a conventional manner used to make pharmaceutical compositions, e.g., by dissolving or suspending the active ingredients in a vehicle such as water for injection with a naturally occurring vegetable oil such as sesame oil and coconut oil, etc. to prepare the pharmaceutical composition. Examples of an aqueous medium for injection include physiological saline and an isotonic solution containing glucose and other auxiliary agents (e.g., D-sorbitol, D-mannitol, sodium chloride, etc.), etc. and may be used in combination with an appropriate dissolution aid such as an alcohol (e.g., ethanol), a polyalcohol (e.g., propylene glycol and polyethylene glycol), a nonionic surfactant (e.g., polysorbate 80™ and HCO-50), etc. As an oily medium, for example, sesame oil, soybean oil, etc. may be used, which can be used in combination with a dissolution aid such as benzyl benzoate, benzyl alcohol, etc.

Furthermore, the prophylactic/therapeutic agent described above may also be formulated with a buffer (e.g., phosphate buffer and sodium acetate buffer) a soothing agent (e.g., benzalkonium chloride, procaine hydrochloride, etc.), a stabilizer (e.g., human serum albumin, polyethylene glycol, etc.), a preservative (e.g., benzyl alcohol, phenol, etc.), an antioxidant, etc. The thus-prepared liquid for injection is normally filled in an appropriate ampoule.

Since the thus obtained pharmaceutical preparation is safe and low toxic, the preparation can be administered to human or mammals (e.g., rat, mouse, rabbit, sheep, swine, bovine, cat, dog, monkey, etc.).

The dose of the receptor protein of the present invention varies depending on subject to be administered, target organ, symptom, route for administration, etc.;

in oral administration, the dose is normally about 0.1 mg to about 100 mg, preferably about 1.0 to about 50 mg, and more preferably about 1.0 to about 20 mg per day for the patient with hypertension (as 60 kg body weight). In parenteral administration, the single dose varies depending on subject to be administered, target organ, symptom, route for administration, etc. but it is advantageous to administer the active ingredient intravenously in a daily dose of about 0.01 to about 30 mg, preferably about 0.1 to about 20 mg, and more preferably about 0.1 to about 10 mg for the patient with hypertension (as 60 kg body weight). For other animal species, the corresponding dose as converted per 60 kg weight can be administered.

The dose of the DNA of the present invention varies depending on subject to be administered, target organ, symptom, route for administration, etc.; in oral administration, the dose is normally about 0.1 mg to about 100 mg, preferably about 1.0 to about 50 mg, and more preferably about 1.0 to about 20 mg per day for the patient with hypertension (as 60 kg body weight). In parenteral administration, the single dose varies depending on subject to be administered, target organ, symptom, route for administration, etc. but it is advantageous to administer the active ingredient intravenously at a daily dose of about 0.01 to about 30 mg, preferably about 0.1 to about 20 mg, and more preferably about 0.1 to about 10 mg for the patient with hypertension (as 60 kg body weight). For other animal species, the corresponding dose as converted per 60 kg weight can be administered.

(3) Gene Diagnostic Agent

Using as a probe the DNA of the present invention, an abnormality (gene abnormality) of the DNA or mRNA encoding the receptor protein of the present invention or its partial peptide in human or mammal (e.g., rat, mouse, rabbit, sheep, swine, bovine, cat, dog, monkey, etc.) can be detected. Therefore, the DNA of the present invention is useful as a gene diagnostic agent for the damage to the DNA or mRNA, mutation thereof, or decreased expression thereof, or increased expression or overexpression of the DNA or mRNA.

The gene diagnosis described above using the DNA of the present invention can be performed by, for example, publicly known northern hybridization assay or PCR-SSCP assay (Genomics, 5, 874-879 (1989); Proceedings of the National Academy of Sciences of the United States of America, 86, 2766-2770 (1989)), etc.

(4) Methods for Screening of Compounds that Alter the Expression Level of the Receptor Protein of the Present Invention or its Partial Peptide

By using the DNA of the present invention as a probe, the DNA can be used for screening of compounds that alter the expression level of the receptor protein of the present invention or its partial peptide.

That is, the present invention provides methods for screening of compounds that alter the expression level of the receptor protein of the present invention or its partial peptide, which comprises measuring the amount of mRNA in the receptor protein of the present invention or its partial peptide contained, for example, in (i) (1) blood, (2) particular organs, (3) tissues or cells isolated from the organs of non-human mammals, or in (ii) transformants, etc.

The amount of mRNA in the receptor protein of the present invention or its partial peptide can be specifically measured as follows.

(i) Normal or disease models of non-human mammals (e.g., mice, rats, rabbits, sheep, swine, bovine, cats, dogs, monkeys, etc., more specifically, rats with dementia, obese mice, rabbits with arteriosclerosis, tumor-bearing mice, etc.) receive administration of a drug (e.g., anti-dementia agents, hypotensive agents, anticancer agents, antiobestic agents, etc.) or physical stress (e.g., soaking stress, electric shock, light, darkness, low temperature, etc.), and blood or particular organs (e.g., brain, liver, kidneys, etc.), or tissues or cells isolated from the organs are obtained after a specified period of time.

The mRNA of the receptor protein of the present invention or its partial peptide contained in the cells thus obtained is extracted from the cells, for example, in a conventional manner and quantified using, e.g., TaqMan PCR, or may also be analyzed by the northern blotting technique by publicly known methods.

(ii) Transformants that express the receptor protein of the present invention or its partial peptide are prepared by the methods described above, and mRNA of the receptor protein of the present invention or its partial peptide can be quantified and analyzed, as described above.

The compounds that alter the expression level of the receptor protein of the present invention or its partial peptide can be screened by the following procedures.

(i) To normal or disease models of non-human mammals, a test compound is administered at a specified period of time before (30 minutes to 24 hours before, preferably 30 minutes to 12 hours before, more preferably 1 hour to 6 hours before), or at a specified time after (30 minutes to 3 days after, preferably 1 hour to 2 days after, more preferably 1 hour to 24 hours after), or simultaneously with a drug or physical stress. At a specified time (30 minute to 3 days, preferably 1 hour to 2 days, more preferably 1 hour to 24 hours) after the adimistration of a test compound, the amount of mRNA in the receptor protein of the present invention or its partial peptide contained in cells are quantified and analyzed.

(ii) While transformants are cultured in a conventional manner, a test compound is added to the culture medium and cultured for a given period of time. After a specified time (1 day to 7 days after, preferably 1 day to 3 days after, more preferably 2 to 3 days after), the amount of mRNA in the receptor protein of the present invention or its partial peptide contained in the transformants are quantified and analyzed.

The compounds or salts thereof, which are obtainable by the screening methods of the present invention, are compounds having the activity of altering the expression level of the receptor protein of the present invention or its partial peptide.

Specifically, they are (a) compounds that potentiate the G protein-coupled receptor-mediated cell stimulating activities (e.g., activities that promote or suppress arachidonic acid release, acetylcholine release, intracellular Ca²⁺ release, intracellular cAMP production, intracellular cGMP production, inositol phosphate production, alters in cell membrane potential, phosphorylation of intracellular proteins, activation of c-fos, pH reduction, etc.) by increasing the expression level of the receptor protein of the present invention or its partial peptide; and (b) compounds that decrease the cell-stimulating activities by reducing the expression level of the receptor protein of the present invention or its partial peptide.

The compounds include peptides, proteins, non-peptide compounds, synthetic compounds, and fermentation products. They may be novel or known compounds.

The compounds that increase the cell-stimulating activities are useful as safe and low-toxic pharmaceuticals for potentiation of the physiological activity of the receptor protein or the like of the present invention.

The compounds that decrease the cell-stimulating activities are useful as safe and low-toxic pharmaceuticals for reducing the physiological activity of the receptor protein or the like of the present invention.

When the compounds or salts thereof, which are obtainable by the screening methods of the present invention, are used in pharmaceutical compositions, the compounds can be formulated by conventional means. For example, as described for the pharmaceuticals containing the receptor protein of the present invention, the compounds can be prepared into tablets, capsules, elixir, microcapsules, aseptic solutions, suspensions, etc.

The preparations obtained as described above are safe and low toxic, and can be administered to human and mammals (e.g., rats, rabbits, sheep, swine, bovine, cats, dogs, monkeys, etc.).

The dose of the compounds or salts thereof varies depending on subject to be administered, target organs, symptom, routes for administration, etc.; in oral administration, the dose is normally about 0.1 to about 100 mg, preferably about 1.0 to about 50 mg, more preferably about 1.0 to about 20 mg per day for the patient with hypertension (as 60 kg body weight). In parenteral administration, the single dose varies depending on subject to be administered, target organ, symptom, method for administration, etc. but it is advantageous to administer the active ingredient intravenously at a daily dose of about 0.01 to about 30 mg, preferably about 0.1 to about 20 mg, more preferably about 0.1 to about 10 mg for the patient with hypertension (as 60 kg body weight). For other animal species, the corresponding dose as converted per 60 kg weight can be administered.

(5) Prophylactic and/or Therapeutic Agents for Various Diseases Containing the Compounds that Alter the Expression Level of the Receptor Protein of the Present Invention or its Partial Peptide

As described above, the receptor protein of the present invention is considered to play some important role in vivo, including, e.g., the central function, etc. Therefore, the compounds that alter the expression level of the receptor protein of the present invention or its partial peptide can be used as prophylactic and/or therapeutic agents for diseases associated with dysfunction of the receptor protein of the present invention.

Where these compounds are used as prophylactic and/or therapeutic agents for diseases associated with dysfunction of the receptor protein of the present invention, the preparations can be obtained by a conventional means.

For example, the compounds can be administered orally in the form of tablets which, if necessary, may be sugar coated, capsules, elixir, microcapsules, etc., or parenterally in the form of injectable preparations such as a sterile solution, a suspension, etc. in water or with other pharmaceutically acceptable liquid. For example, these preparations can be manufactured by mixing with physiologically acceptable known carriers, flavors, fillers, vehicles, antiseptics, stabilizers, binders, etc. in a unit dosage form required for generally approved pharmaceutical preparations. The active ingredient in the preparation is controlled in such a dose that an appropriate dose is obtained within the specified range given.

Additives miscible with tablets, capsules etc. include a binder such as gelatin, corn starch, tragacanth and gum arabic, an excipient such as crystalline cellulose, a swelling agent such as corn starch, gelatin, alginic acid, etc., a lubricant such as magnesium stearate, a sweetening agent such as sucrose, lactose and saccharin, and a flavoring agent such as peppermint, akamono oil and cherry. When the unit dosage is in the form of capsules, liquid carriers such as oils and fats may further be used together with the additives described above. A sterile composition for injection may be formulated according to a conventional manner used to make pharmaceutical compositions, e.g., by dissolving or suspending the active ingredients in a vehicle such as water for injection with a naturally occurring vegetable oil such as sesame oil and coconut oil, etc. to prepare the pharmaceutical composition. Examples of an aqueous medium for injection include physiological saline and an isotonic solution containing glucose and other auxiliary agents (e.g., D-sorbitol, D-mannitol, sodium chloride, etc.), etc. and may be used in combination with an appropriate dissolution aid such as an alcohol (e.g., ethanol), a polyalcohol (e.g., propylene glycol and polyethylene glycol), a nonionic surfactant (e.g., polysorbate 80™ and HCO-50), etc. As an oily medium, for example, sesame oil, soybean oil, etc. may be used, which can be used in combination with a dissolution aid such as benzyl benzoate, benzyl alcohol, etc.

Furthermore, the prophylactic/therapeutic agent described above may also be formulated with a buffer (e.g., phosphate buffer and sodium acetate buffer) a soothing agent (e.g., benzalkonium chloride, procaine hydrochloride, etc.), a stabilizer (e.g., human serum albumin, polyethylene glycol, etc.), a preservative (e.g., benzyl alcohol, phenol, etc.), an antioxidant, etc. The thus-prepared liquid for injection is normally filled in an appropriate ampoule.

The preparations obtained as described above are safe and low toxic, and can be administered to, for example, humans and mammals (e.g., rats, rabbits, sheep, swine, bovine, cats, dogs, monkeys, etc.).

The dose of the compounds or salts thereof varies depending on subject to be administered, target organs, symptom, routes for administration, etc.; in oral administration, the dose is normally about 0.1 mg to about 100 mg, preferably about 1.0 to about 50 mg, and more preferably about 1.0 to about 20 mg per day for the patient with hypertension (as 60 kg body weight). In parenteral administration, the single dose varies depending on subject to be administered, target organ, symptom, route for administration, etc. but it is advantageous to administer the active ingredient intravenously in a daily dose of about 0.01 to about 30 mg, preferably about 0.1 to about 20 mg, and more preferably about 0.1 to about 10 mg for the patient with hypertension (as 60 kg body weight). For other animal species, the corresponding dose as converted per 60 kg weight can be administered.

(6) Quantification of a Ligand to the G Protein-Coupled Receptor Protein of the Present Invention

Since the receptor protein or the like of the present invention has a binding property to a ligand, the ligand activity can be quantified in vivo with high sensitivity.

The method of quantification of the present invention may be effected, for example, in combination with a competitive method. That is, a test sample to be determined is brought into contact with the receptor protein or the like of the present invention, whereby the ligand concentration in the test sample can be determined. Specifically, the quantification can be performed by the following method (1) or (2) below or its modifications:

(1) Hiroshi Irie (ed.): “Radioimmunoassay” (1974, published by Kodansha, Japan); and

(2) Hiroshi Irie (ed.): “Radioimmunoassay, Second Series” (1979, published by Kodansha, Japan,).

(7) Methods for Screening of the Compounds (Agonists, Antagonists, etc.) that Alter the Binding Property Between the G Protein-Coupled Receptor Protein of the Present Invention and a Ligand

By using the receptor protein or the like of the present invention or salts thereof, or by using the receptor-binding assay system via the constructed expression system of a recombinant receptor protein, etc., screening can be made efficiently on the compounds (e.g., peptides, proteins, non-peptide compounds, synthetic compounds, fermentation products, etc.) or salts thereof that alter the binding property between a ligand and the receptor protein or the like of the present invention.

Examples of such compounds include (a) compounds exhibiting the G protein-coupled receptor-mediated cell stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.) (so-called agonists to the receptor protein of the present invention), (b) compounds having no such cell stimulating activities (so-called antagonists to the receptor protein of the present invention); (c) compounds that potentiate the binding force between a ligand and the G protein-coupled receptor protein of the present invention; or (d) compounds that decrease the binding force between a ligand and the G protein-coupled receptor protein of the present invention; etc. (the compounds (a) are screened preferably by the methods of determining a ligand described above).

That is, the present invention also provides a method of screening a compound or its salt that alters the binding property between a ligand and the receptor protein of the present invention, its partial peptide or a salt thereof, in which comparison is made between the following cases: (i) the case wherein the receptor protein of the present invention, its partial peptide or a salt thereof is brought in contact with a ligand; and (ii) the case wherein the receptor protein of the present invention, its partial peptide or a salt thereof is brought in contact with a ligand and a test compound.

In the screening methods of the present invention, the amount of the ligand bound to the receptor protein or the like, the cell-stimulating activities, etc. are measured in the cases (i) and (ii) and comparison is made between the two cases.

More specifically, the present invention provides the following methods.

(1) A method of screening a compound or its salt that alters the binding property between a ligand and the receptor protein or the like of the present invention, which comprises measuring the amount of a labeled ligand bound to the receptor protein or the like when the labeled ligand is brought in contact with the receptor protein or the like of the present invention and when the labeled ligand and a test compound are brought in contact with the receptor protein or the like of the present invention, and comparing the binding amounts.

(2) A method of screening a compound or its salt that alters the binding property between a ligand and the receptor protein or the like of the present invention, which comprises measuring the amount of a labeled ligand bound to a cell containing the receptor protein or the like of the present invention or a membrane fraction of the cell, when the labeled ligand is brought in contact with the cell containing the receptor protein or the like of the present invention or the cell membrane fraction and when the labeled ligand and a test compound are brought in contact with the cell containing the receptor protein or the like of the present invention or the cell membrane fraction, and comparing the binding amounts.

(3) A method of screening a compound or its salt that alters the binding property between a ligand and the receptor protein or the like of the present invention, which comprises measuring the amount of a labeled ligand bound to the receptor protein or the like of the present invention, when the labeled ligand is brought in contact with the receptor protein or the like expressed on a cell membrane by culturing a transformant containing the DNA of the present invention and when the labeled ligand and a test compound are brought in contact with the receptor protein or the like expressed on a cell membrane by culturing a transformant containing the DNA of the present invention, and comparing the binding amounts.

(4) A method of screening a compound or its salt that alters the binding property between a ligand and the receptor protein or the like of the present invention, which comprises measuring the receptor-mediated cell stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.), when a compound (e.g., a ligand to the receptor protein or the like of the present invention) that activates the receptor protein or the like of the present invention is brought in contact with a cell containing the receptor protein or the like of the present invention and when a compound that activates the receptor protein or the like of the present invention and a test compound are brought in contact with the cell containing the receptor protein or the like of the present invention, and comparing the cell stimulating activities.

(5) A method of screening a compound or its salt that alters the binding property between a ligand and the receptor protein or the like of the present invention, which comprises measuring the receptor-mediated cell stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.), when a compound that activates the receptor protein or the like of the present invention (e.g., a ligand to the receptor protein or the like of the present invention) is brought in contact with the receptor protein or the like of the present invention expressed on a cell membrane by culturing a transformant containing the DNA of the present invention and when a compound that activates the receptor protein or the like of the present invention and a test compound are brought in contact with the receptor protein or the like of the present invention expressed on a cell membrane by culturing a transformant containing the DNA of the present invention, and comparing the cell stimulating activities.

Before the receptor protein or the like of the present invention was obtained, it was required to screen G protein-coupled receptor agonists or antagonists by acquiring candidate compounds first using cells or tissues containing the G protein-coupled receptor protein or the cell membrane fraction from rats or other animals (primary screening), and then examining the candidate compounds whether the compounds actually inhibit the binding between human G protein-coupled receptor protein and ligands (secondary screening). When cells, tissues or cell membrane fractions were directly used, it was practically difficult to screen agonists or antagonists to the objective receptor protein, since other receptor proteins were present together.

However, the use of, e.g., the human-derived receptor protein of the present invention requires no primary screening but enables to efficiently screen the compounds that inhibit the binding between a ligand and the G protein-coupled receptor protein. Besides, it is easy to assess whether the screened compound is either an agonist or an antagonist.

Hereinafter the screening method of the present invention will be described specifically.

First, the receptor protein or the like of the present invention, which is used for the screening methods of the present invention, may be any protein, so long as it contains the receptor protein or the like of the present invention described above, though cell membrane fractions of mammalian organs are preferably employed. For screening, however, it is advantageous to use human-derived receptor proteins or the like expressed abundantly using recombinants, especially because it is extremely difficult to make the organs of human origin available.

In the manufacture of the receptor protein or the like of the present invention, the methods described above can be used, though it is preferred to manufacture the receptor protein or the like through expression of the DNA of the present invention in mammalian cells or insect cells. As the DNA fragment encoding the target protein region, complementary DNA may be used but is not limited thereto. For example, gene fragments or synthetic DNA may also be used as the DNA fragment. In order to introduce the DNA fragment encoding the receptor protein of the present invention into host animal cells and express it efficiently, the DNA fragment is preferably incorporated into a polyhedron promoter of nuclear polyhedrosis virus (NPV) belonging to the baculovirus, a SV40-derived promoter, a promoter of retrovirus, a metallothionein promoter, a human heat shock promoter, a cytomegalovirus promoter, SRα promoter, etc. at the downstream thereof. The quantity and quality of the thus expressed receptors can be examined by a publicly known method, for example, by the method described in the literature [Nambi, P. et al., J. Biol. Chem., 267, 19555-19559, 1992].

Accordingly, in the screening methods of the present invention, the substances containing the receptor protein or the like of the present invention may be the receptor protein or the like that is purified by publicly known methods. Alternatively, cells containing the receptor protein or the like or membrane fractions of the cells containing the receptor protein or the like may be used as well.

Where the cells containing the receptor protein or the like of the present invention are used in the screening method of the present invention, these cells may be fixed with glutaraldehyde, formalin, etc. The fixation may be carried out by a publicly known method.

The cells containing the receptor protein or the like of the present invention refer to host cells expressing the receptor protein or the like. Examples of such host cells include Escherichia coli, Bacillus subtilis, yeast, insect cells and animal cells.

The cell membrane fraction refers to a fraction that abundantly contains the cell membranes prepared by a publicly known method after disrupting the cells. Examples of cell disruption include cell squashing using a Potter-Elvehjem homogenizer, disruption using a Waring blender or Polytron (manufactured by Kinematica Inc.), disruption by ultrasonication, and disruption by cell spraying via a thin nozzle under increased pressure using a French press or the like. Cell membrane fractionation is effected mainly by fractionation using a centrifugal force, such as centrifugation for fractionation and density gradient centrifugation. For example, cell disruption fluid is centrifuged at a low rate (500 rpm to 3,000 rpm) for a short period of time (normally about 1 to 10 minutes), the resulting supernatant is then centrifuged at a higher rate (15,000 rpm to 30,000 rpm) normally for 30 minutes to 2 hours. The precipitate thus obtained is used as the membrane fraction. The membrane fraction is rich in the receptor protein or the like expressed and membrane components such as cell-derived phospholipids and membrane proteins.

The amount of the receptor protein contained in the cells containing the receptor protein or the like or in the membrane fractions is preferably 10³to 10⁸ molecules per cell, more preferably 10⁵ to 10⁷ molecules per cell. As the amount of expression increases, the ligand binding activity per unit of membrane fraction (specific activity) increases so that not only the highly sensitive screening system can be constructed but also large quantities of samples can be assayed with the same lot.

To perform the methods (1) through (3) for screening the compound that alters the binding property between a ligand and the receptor protein or the like of the present invention, an appropriate receptor protein fraction and a labeled ligand are required.

The receptor protein fraction is preferably a fraction of naturally occurring receptor protein or a recombinant receptor protein fraction having an activity equivalent to that of the naturally occurring protein. Herein, the term equivalent activity is intended to mean a ligand binding activity, a signal transduction activity, etc., equivalent to that of naturally occurring receptor proteins.

As the labeled ligand, a labeled ligand, a compound analogous to the labeled ligand, etc. are employed. Examples of the labeled ligand include ligands labeled with [³H], [¹²⁵I], [¹⁴C], [³⁵S], etc.

Specifically, the compound that alters the binding property between a ligand and the receptor protein or the like of the present invention is screened by the following procedures. First, a receptor protein preparation is prepared by suspending cells containing the receptor protein or the like of the present invention or membrane fractions of the cells in a buffer appropriate for use in the screening methods. Any buffer can be used so long as it does not interfere the ligand-receptor protein binding. Examples of such buffers are a phosphate buffer, a Tris-HCI buffer, etc. having pH of 4 to 10 (preferably pH of 6 to 8). For the purpose of reducing a non-specific binding, a surfactant such as CHAPS, Tween-80™ (Kao-Atlas Inc.), digitonin, deoxycholate, etc. may be added to buffers. Moreover, for the purpose of preventing the degradation of receptors or ligands by a protease, protease inhibitors such as PMSF, leupeptin, E-64 (manufactured by Peptide Institute, Inc.), pepstatin, etc. may also be added. A given amount (5,000 cpm to 500,000 cpm) of a labeled ligand is added to 0.01 ml to 10 ml of the receptor solution, in which 10⁻⁴ M to 10⁻¹⁰ M of a test compound is co-present. To determine the amount of a non-specific binding (NSB), a reaction tube containing an unlabeled ligand in a large excess is also provided. The reaction is carried out at approximately 0° C. to 50° C., preferably about 4° C. to 37° C. for about 20 minutes to about 24 hours, preferably about 30 minutes to 3 hours. After completion of the reaction, the reaction mixture is filtrated through glass fiber filter paper, etc. and washed with an appropriate amount of the same buffer. The residual radioactivity in the glass fiber filter paper is then measured by means of a liquid scintillation counter or γ-counter. When nonspecific binding (NSB) is subtracted from the count (B₀) where any antagonizing substance is absent and the resulting count (B₀−NSB) is made 100%, a test compound showing the specific binding amount (B−NSB) of, e.g., 50% or less may be selected as a candidate compound having an antagonizing inhibition activity.

The method (4) or (5) above for screening the compound that alters the binding property between a ligand and the receptor protein or the like of the present invention can be performed as follows. For example, the receptor protein-mediated cell stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.) may be determined by publicly known methods, or using assay kits commercially available.

Specifically, the cells containing the receptor protein or the like of the present invention are first cultured on a multiwell plate, etc. Prior to screening, the medium is replaced with fresh medium or with an appropriate non-cytotoxic buffer, followed by incubation for a given period of time in the presence of a test compound, etc. Subsequently, the cells are extracted or the supernatant is recovered and the resulting product is quantified by the respective procedures. Where it is difficult to detect the production of the cell-stimulating activity indicator substance (e.g., arachidonic acid, etc.) due to a degrading enzyme contained in the cells, an inhibitor against such a degrading enzyme may be added prior to the assay. For detecting activities such as the cAMP production suppression activity, the baseline production in the cells is increased by forskolin or the like and the suppressing effect on the increased baseline production can then be detected.

For screening by assaying the cell stimulating activities, cells in which an appropriate receptor protein has been expressed are necessary. Preferred cells in which the receptor protein or the like of the present invention has been expressed are a naturally occurring cell line containing the receptor protein or the like of the present invention, the aforesaid cell line in which the recombinant type receptor protein or the like has been expressed, and the like.

Examples of the test compounds include peptides, proteins, non-peptide compounds, synthetic compounds, fermentation products, cell extracts, plant extracts, animal tissue extracts, etc. These compounds may be either novel or publicly known compounds.

The kits for screening of the compound or its salt that alters the binding property between a ligand and the receptor protein or the like of the present invention comprise the receptor protein or the like of the present invention, cells containing the receptor protein or the like of the present invention, or a membrane fraction of the cells containing the receptor protein or the like of the present invention, and the like.

Examples of the screening kits include as follows:

1. Reagent for screening

(1) Assay and Wash Buffers

Hanks' Balanced Salt Solution (manufactured by Gibco Co.) supplemented with 0.05% bovine serum albumin (manufactured by Sigma Co.).

The solution is sterilized by filtration through a 0.45 μm filter and stored at 4° C. Alternatively, the solution may be prepared at use.

(2) G Protein-Coupled Receptor Protein Preparation

CHO cells on which the receptor protein of the present invention has been expressed are subcultured in a 12-well plate at the rate of 5×10⁵ cells/well and then cultured at 37° C. under 5% CO₂ and 95% air for 2 days.

(3) Labeled Ligand

A ligand labeled with commercially available [³H], [¹²⁵I], [¹⁴C], [³⁵S], etc., or a compound labeled by appropriate methods.

An aqueous solution of the compound is stored at 4° C. or −20° C. The solution is diluted to 1 μM with an assay buffer at use. A sparingly water-soluble test compound is dissolved in dimethylformamide, DMSO or methanol.

(4) Ligand Standard Solution

A ligand is dissolved in PBS containing 0.1% bovine serum albumin (manufactured by Sigma Co.) in a concentration of 1 mM, which solution is stored at −20° C.

2. Assay Method

(1) CHO cells of expressing the receptor protein of the present invention cultured in a 12-well tissue culture plate are washed twice with 1 ml of assay the assay buffer, 490 μl of the assay buffer is added to each well.

(2) After 5 μl of a test compound solution of 10⁻³ to 10⁻¹⁰ M is added, 5 μl of a labeled ligand is added to the system followed by incubating at room temperature for an hour. To determine the amount of the non-specific binding, 5 μl of the ligand of 10⁻³ M is added to the system, instead of the test compound.

(3) The reaction mixture is removed from the well, which is washed three times with 1 ml each of the assay buffer. The labeled ligand bound to the cells is dissolved in 0.2N NaOH-1% SDS and mixed with 4 ml of a liquid scintillator A (manufactured by Wako Pure Chemical Industries, Ltd.).

(4) Radioactivity is measured using a liquid scintillation counter (manufactured by Beckmann) and PMB (percent of the maximum binding) is calculated in accordance with the following equation [1]:

PMB =[(B−NSB)/(B₀−NSB)]×100

wherein:

| | |
|---|---|
| PMB: | percent maximum binding |
| B: | value when a sample is added |
| NSB: | non-specific binding |
| B_{0:} | maximum binding |

The compounds or salts thereof obtainable using the screening methods or by the screening kits of the present invention are compounds that function to alter the binding property between ligands and the receptor protein or the like of the present invention. Specifically, these compounds include (a) compounds exhibiting the G protein-coupled receptor-mediated cell stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.) (so-called agonists to the receptor protein of the present invention), (b) compounds having no such cell stimulating activities (so-called antagonists to the receptor protein of the present invention); (c) compounds that potentiate the binding force between ligands and the G protein-coupled receptor protein of the present invention and (d) compounds that reduce the binding force between ligands and the G protein-coupled receptor protein of the present invention.

Examples of such compounds include peptides, proteins, non-peptide compounds, synthetic compounds and fermentation products. These compounds may be either novel or publicly known compounds.

The agonists to the receptor protein or the like of the present invention have similar physiological activities to those possessed by the receptor protein or the like of the present invention, and are thus useful as safe and low toxic pharmaceuticals, depending on the ligand activities.

The antagonists to the receptor protein or the like of the present invention can suppress the physiological activities possessed by the receptor protein or the like of the present invention, and are thus useful as safe and low toxic pharmaceuticals for suppressing the ligand activities. The compounds that increase the binding force between ligands and the G protein-coupled receptor protein of the present invention are useful as safe and low toxic pharmaceuticals for potentiation of the physiological activities possessed by the ligands to the receptor protein or the like of the present invention has.

The compounds that decrease the binding force between ligands and the G protein-coupled receptor protein of the present invention are useful as safe and low toxic pharmaceuticals for reducing the physiological activities possessed by the ligands to the receptor protein or the like of the present invention.

When the compounds or salts thereof obtainable using the screening methods or the screening kits of the present invention are used for the pharmaceutical preparations described above, a conventional means may be applied to making pharmaceutical preparations. For example, the compounds or salt thereof may be prepared in the form of tablets, capsules, elixir, microcapsules, sterile solutions, suspensions, etc., like the aforesaid pharmaceuticals containing the receptor protein of the present invention.

Since the thus obtained preparations are safe and low toxic, they can be administered to, for example, human or mammals (e.g., rat, mouse, rabbit, sheep, swine, bovine, cat, dog, monkey, etc.).

The dose of the compound or its salt varies depending on subject to be administered, target organ, symptom, route for administration, etc.; in oral administration, the dose is normally about 0.1 to about 100 mg, preferably about 1.0 to about 50 mg, more preferably about 1.0 to about 20 mg per day for the patient with hypertension (as 60 kg body weight). In parenteral administration, the single dose varies depending on subject to be administered, target organ, symptom, method for administration, etc. but it is advantageous to administer the active ingredient intravenously at a daily dose of about 0.01 to about 30 mg, preferably about 0.1 to about 20 mg, more preferably about 0.1 to about 10 mg for the patient with hypertension (as 60 kg body weight). For other animal species, the corresponding dose as converted per 60 kg weight can be administered.

(8) Prophylactic and/or Therapeutic Agents for Various Diseases Comprising the Compounds (Agonists, Antagonists) that Alter the Binding Property Between the G Protein-Coupled Receptor Protein of the Present Invention and Ligands

As stated hereinabove, the receptor protein of the present invention plays some important role in vivo, such as the central function, etc. Therefore, the compounds (agonists, antagonists) that alter the binding property between the receptor protein of the present invention and a ligand can be used as the prophylactic and/or therapeutic agents of diseases associated with dysfunction of the receptor protein of the present invention.

Where the compounds above are used as the prophylactic and/or therapeutic agents of diseases associated with dysfunction of the receptor protein of the present invention, a conventional means may be applied to making pharmaceutical preparations.

For example, the compounds may be prepared in the form of tablets which, if necessary, may be sugar coated, capsules, elixir, microcapsules, etc., for oral administration and for parenteral administration in the form of injectable preparations such as a sterile solution and a suspension in water or with other pharmaceutically acceptable liquid. These preparations can be manufactured by mixing the compound with a physiologically acceptable known carrier, a flavoring agent, an excipient, a vehicle, an antiseptic, a stabilizer, a binder, etc. in a unit dosage form required in a generally accepted manner for making pharmaceutical preparations. The active ingredient in the preparation is controlled in such a dose that an appropriate dose is obtained within the specified range given.

Additives miscible with tablets, capsules etc. include a binder such as gelatin, corn starch, tragacanth and gum arabic, an excipient such as crystalline cellulose, a swelling agent such as corn starch, gelatin, alginic acid, etc., a lubricant such as magnesium stearate, a sweetening agent such as sucrose, lactose and saccharin, and a flavoring agent such as peppermint, akamono oil and cherry. When the unit dosage is in the form of capsules, liquid carriers such as oils and fats may further be used together with the additives described above. A sterile composition for injection may be formulated according to a conventional manner used to make pharmaceutical compositions, e.g., by dissolving or suspending the active ingredients in a vehicle such as water for injection with a naturally occurring vegetable oil such as sesame oil and coconut oil, etc. to prepare the pharmaceutical composition. Examples of an aqueous medium for injection include physiological saline and an isotonic solution containing glucose and other auxiliary agents (e.g., D-sorbitol, D-mannitol, sodium chloride, etc.), etc. and may be used in combination with an appropriate dissolution aid such as an alcohol (e.g., ethanol), a polyalcohol (e.g., propylene glycol and polyethylene glycol), a nonionic surfactant (e.g., polysorbate 80™ and HCO-50), etc. As an oily medium, for example, sesame oil, soybean oil, etc. may be used, which can be used in combination with a dissolution aid such as benzyl benzoate, benzyl alcohol, etc.

Furthermore, the prophylactic/therapeutic agent described above may also be formulated with a buffer (e.g., phosphate buffer and sodium acetate buffer) a soothing agent (e.g., benzalkonium chloride, procaine hydrochloride, etc.), a stabilizer (e.g., human serum albumin, polyethylene glycol, etc.), a preservative (e.g., benzyl alcohol, phenol, etc.), an antioxidant, etc. The thus prepared liquid for injection is normally filled in an appropriate ampoule.

The pharmaceutical preparation thus obtained is safe and low toxic, and can be administered, for example, to human or mammals (e.g., rat, mouse, rabbit, sheep, swine, bovine, cat, dog, monkey, etc.).

The dose of the compound or its salt varies depending on subject to be administered, target organ, symptom, route for administration, etc.; in oral administration, the dose is normally about 0.1 to about 100 mg, preferably about 1.0 to about 50 mg, and more preferably about 1.0 to about 20 mg per day for the patient with hypertension (as 60 kg body weight). In parenteral administration, the single dose varies depending on subject to be administered, target organ, symptom, route for administration, etc. but it is advantageous to administer the active ingredient intravenously in a daily dose of about 0.01 to about 30 mg, preferably about 0.1 to about 20 mg, and more preferably about 0.1 to about 10 mg for the patient with hypertension (as 60 kg body weight). For other animal species, the corresponding dose as converted per 60 kg weight can be administered.

(9) Quantification of the Receptor Protein of the Present Invention or its Partial Peptide, or Salts Thereof

The antibody of the present invention is capable of specifically recognizing the receptor protein or the like of the present invention and accordingly, can be used for quantification of the receptor protein or the like of the present invention in a test sample, in particular, for quantification by sandwich immunoassay. That is, the present invention provides, for example, (i) a method of quantification of the receptor protein or the like of the present invention in a test sample, which comprises competitively reacting the antibody of the present invention, a test sample and a labeled form of the receptor protein or the like of the present invention, and measuring the ratio of the labeled receptor protein or the like bound to the antibody; and, (ii) a method of quantification of the receptor protein or the like of the present invention in a test sample, which comprises simultaneously or continuously reacting a test sample with the antibody of the present invention immobilized on an insoluble carrier and a labeled form of the antibody of the present invention, and measuring the activity of the labeling agent on the insoluble carrier.

In the method (ii) described above, it is preferred that one antibody is capable of recognizing the N-terminal region of the receptor protein or the like of the present invention, while another antibody is capable of recognizing the C-terminal region of the receptor protein or the like of the present invention.

The monoclonal antibody to the receptor protein or the like of the present invention (hereinafter sometimes merely referred to as the monoclonal antibody of the present invention) may be used to assay the receptor protein or the like of the present invention. Moreover, the receptor protein or the like of the present invention can be detected by means of a tissue staining as well. For these purposes, the antibody molecule per se may be used or F(ab′)₂, Fab′ or Fab fractions of the antibody molecule may also be used. There is no particular limitation for the assaying method using the antibody to the receptor protein of the present invention; any method may be used so far as it relates to a method in which the amount of antibody, antigen or antibody-antigen complex can be detected by a chemical or a physical means, depending on or corresponding to the amount of antigen (e.g., the amount of the receptor protein) in a test sample to be assayed, and then calculated using a standard curve prepared by a standard solution containing the known amount of antigen. Advantageously used are, for example, nephrometry, competitive method, immunometric method and sandwich method; in terms of sensitivity and specificity, the sandwich method, which will be described later, is particularly preferred.

Examples of the labeling agents used in the assay methods using labeling substances are radioisotopes, enzymes, fluorescent substances, luminescent substances, etc. Examples of the radioisotope are [¹²⁵I], [¹³¹I], [³H], [¹⁴C], etc. Preferred examples of the enzyme are those that are stable and have a high specific activity, which include β-galactosidase, β-glucosidase, alkaline phosphatase, peroxidase and malate dehydrogenase. Examples of the fluorescent substance are fluorescamine, fluorescein isothiocyanate, etc. Examples of the luminescent substance are luminol, a luminol derivative, luciferin, lucigenin, etc. Furthermore, a biotin-avidin system may also be used for binding an antibody or antigen to a labeling agent.

In the immobilization of antigens or antibodies, physical adsorption may be used. Alternatively, chemical binding that is conventionally used for immobilization of proteins or enzymes may be used as well. Examples of the carrier include insoluble polysaccharides such as agarose, dextran and cellulose; synthetic resins such as polystyrene, polyacrylamide and silicone; glass; etc.

In the sandwich method, a test sample liquid is reacted with an immobilized monoclonal antibody of the present invention (primary reaction), then reacted with a labeled form of the monoclonal antibody of the present invention (secondary reaction) and the activity of the labeling agent on the insoluble carrier is assayed, whereby the amount of the receptor protein of the present invention in the test sample liquid can be determined. The primary and secondary reactions may be carried out in a reversed order, simultaneously or sequentially with an interval. The type of the labeling agent and the method for immobilization may be the same as those described hereinabove.

In the immunoassay by the sandwich method, it is not always necessary that the antibody used for the labeled antibody and for the solid phase should be one type or one species but a mixture of two or more antibodies may also be used for the purpose of improving the measurement sensitivity, etc.

In the method of assaying the receptor protein or the like by the sandwich method according to the present invention, the monoclonal antibodies of the present invention used for the primary and secondary reactions are preferably antibodies, which binding sites to the receptor protein or the like are different from each other. Thus, the antibodies used in the primary and secondary reactions are those wherein, when the antibody used in the secondary reaction recognizes the C-terminal region of the receptor protein, the antibody recognizing the site other than the C-terminal regions, e.g., recognizing the N-termiinal region, is preferably used in the primary reaction.

The monoclonal antibody of the present invention may be used in an assay system other than the sandwich method, such as a competitive method, an immunometric method and a nephrometry. In the competitive method, an antigen in a test solution and a labeled antigen are competitively reacted with an antibody, then an unreacted labeled antigen (F) and a labeled antigen bound to the antibody (B) are separated (B/F separation) and the labeled amount of either B or F is measured to determine the amount of the antigen in the test solution. In the reactions for such a method, there are a liquid phase method in which a soluble antibody is used as the antibody and the B/F separation is effected by polyethylene glycol while a second antibody to the antibody is used, and a solid phase method in which an immobilized antibody is used as the first antibody or a soluble antibody is used as the first antibody while an immobilized antibody is used as the second antibody.

In the immunometric method, an antigen in a test solution and an immobilized antigen are competitively reacted with a given amount of a labeled antibody followed by separating the solid phase from the liquid phase; or an antigen in a test solution and an excess amount of labeled antibody are reacted, then an immobilized antigen is added to bind an unreacted labeled antibody to the solid phase and the solid phase is separated from the liquid phase. Thereafter, the labeled amount of any of the phases is measured to determine the antigen amount in the test solution.

In the nephrometry, the amount of insoluble sediment, which is produced as a result of the antigen-antibody reaction in a gel or in a solution, is measured. Even when the amount of an antigen in a test solution is small and only a small amount of the sediment is obtained, a laser nephrometry utilizing laser scattering can be suitably used.

In applying each of those immunoassays to the assay method of the present invention, any special conditions or operations are not required to set forth. The assay system for the receptor protein of the present invention or salts thereof may be constructed in addition to conditions or operations conventionally used for each of the methods, taking the technical consideration of one skilled in the art into account. For the details of such conventional technical means, a variety of reviews, reference books, etc. may be referred to [see, for example, Hiroshi Irie (ed.): “Radioimmunoassay” (published by Kodansha, 1974); Hiroshi Irie (ed.): “Radioimmunoassay; Second Series” (published by Kodansha, 1979); Eiji Ishikawa, et al. (ed.): “Enzyme Immunoassay” (published by Igaku Shoin, 1978); Eiji Ishikawa, et al. (ed.): “Enzyme Immunoassay” (Second Edition) (published by Igaku Shoin, 1982); Eiji Ishikawa, et al. (ed.): “Enzyme Immunoassay” (Third Edition) (published by Igaku Shoin, 1987); “Methods in Enzymology” Vol. 70 (Immunochemical Techniques (Part A)); ibid., Vol. 73 (Immunochemical Techniques (Part B)); ibid., Vol. 74 (Immunochemical Techniques (Part C)); ibid., Vol. 84 (Immunochemical Techniques (Part D: Selected Immunoassays)); ibid., Vol. 92 (Immunochemical Techniques (Part E: Monoclonal Antibodies and General Immunoassay Methods)); ibid., Vol. 121 (Immunochemical Techniques (Part I: Hybridoma Technology and Monoclonal Antibodies)) (published by Academic Press); etc.]

As described above, the receptor protein of the present invention or salts thereof can be quantified with high sensitivity, using the antibody of the present invention.

Furthermore, the receptor protein of the present invention or salts thereof can be quantified in vivo, using the antibody of the present invention thereby to diagnose various diseases associated with the dysfunction of the receptor protein of the present invention.

The antibody of the present invention can be employed for specifically detecting the receptor protein or the like of the present invention, which may be present in a test sample such as a body fluid, a tissue, etc. The antibody can also be used for preparation of an antibody column for purification of the receptor protein or the like of the present invention, detection of the receptor protein or the like of the present invention in the fractions upon purification, and analysis of the behavior of the receptor protein of the present invention in the cells under investigation.

(10) Methods for Screening of Compounds that Alter the Amount of the Receptor Protein of the Present Invention or its Partial Peptide on Cell Membranes

Since the antibodies of the present invention specifically recognize the receptor protein of the present invention or its partial peptide, or salts thereof, the antibodies can be used to screen the compounds that alter the amount of the receptor protein of the present invention or its partial peptide on cell membranes.

That is, the present invention provides, for example, the following methods:

(i) A method for screening of compounds that alter the amount of the receptor protein of the present invention or its partial peptides in cell membranes, which comprises disrupting (1) blood, (2) particular organs, (3) tissues, cells, etc. isolated from the organs of non-human mammals, isolating the cell membrane fraction and then quantifying the receptor protein of the present invention or its partial peptide contained in the cell membrane fraction;

(ii) A method for screening of compounds that alter the amount of the receptor protein of the present invention or its partial peptides in cell membranes, which comprises disrupting transformants, etc. expressing the receptor protein of the present invention or its partial peptides, isolating the cell membrane fraction, and then quantifying the receptor protein of the present invention or its partial peptides contained in the cell membrane fraction;

(iii) A method for screening of compounds that alter the amount of the receptor protein of the present invention or its partial peptides in cell membranes, which comprises sectioning (1) blood, (2) particular organs, (3) tissues, cells, etc. isolated from the organs of non-human mammals, immunostaining, and then quantifying the staining intensity of the receptor protein on the cell surface layer to confirm the protein on the cell membrane isolating cell membrane fractions, and quantifying the receptor protein on the cell membrane; and,

(iv) A method for screening of compounds that alter the amount of the receptor protein of the present invention or its partial peptides on cell membranes, which comprises sectioning transformants, etc. expressing the receptor protein of the present invention or its partial peptides, immunostaining, and then quantifying the staining intensity of the receptor protein in the cell surface layer to confirm the protein on the cell membrane.

Specifically, the receptor protein and its partial peptides of the present invention contained in cell membrane fractions are quantified as follows.

(i) Normal or disease models of non-human mammals (e.g., mice, rats, rabbits, sheep, swine, bovine, cats, dogs, monkeys, etc., more specifically, rats with dementia, obese mice, rabbits with arteriosclerosis, tumor-bearing mice, etc.) receive administration of a drug (e.g., anti-dementia agents, hypotensive agents, anticancer agents, antiobestic agents, etc.) or physical stress (e.g., soaking stress, electric shock, light, darkness, low temperature, etc.), and blood or particular organs (e.g., brain, liver, kidneys, etc.), or tissues or cells isolated from the organs are obtained after a specified period of time. The organs, tissues, cells, etc. thus obtained are suspended in, for example, an appropriate buffer (e.g., Tris hydrochloride buffer, phosphate buffer, Hepes buffer, etc.), and the organs, tissues or cells are disrupted, and the cell membrane fractions are obtained using surfactants (e.g., Triton-X 100™, Tween 20™, etc.) and further using techniques such as centrifugal separation, filtration, column fractionation, etc.

The cell membrane fraction refers to a fraction abundant in cell membranes, obtained by cell disruption and subsequent fractionation by publicly known methods. Useful cell disruption methods include cell squashing using a Potter-Elvehjem homogenizer, disruption using a Waring blender or Polytron (manufactured by Kinematica Inc.), disruption by ultrasonication, and disruption by cell spraying through thin nozzles under an increased pressure using a French press or the like. Cell membrane fractionation is effected mainly by fractionation using a centrifugal force, such as centrifugation for fractionation and density gradient centrifugation. For example, cell disruption fluid is centrifuged at a low speed (500 rpm to 3,000 rpm) for a short period of time (normally about 1 to about 10 minutes), the resulting supernatant is then centrifuged at a higher speed (15,000 rpm to 30,000 rpm) normally for 30 minutes to 2 hours. The precipitate thus obtained is used as the membrane fraction. The membrane fraction is rich in the receptor protein or the like expressed and membrane components such as cell-derived phospholipids, membrane proteins, etc.

The receptor protein of the present invention or its partial peptides contained in the cell membrane fraction can be quantified by, for example, the sandwich immunoassay and the western blotting analysis using the antibodies of the present invention.

The sandwich immunoassay can be performed as described above, and the western blotting analysis can be performed by publicly known methods.

(ii) Transformants that express the receptor protein of the present invention or its partial peptides are prepared following the methods described above, and the receptor protein of the present invention or its partial peptides contained in the cell membrane fraction can be quantified.

The compounds that alter the amount of the receptor protein of the present invention or its partial peptides in cell membranes can be screened as follows.

(i) To normal or disease models of non-human mammals, a test compound is administered at a specified period of time before (30 minutes to 24 hours before, preferably 30 minutes to 12 hours before, more preferably 1 hour to 6 hours before), at a specified time after (30 minutes to 3 days after, preferably 1 hour to 2 days after, more preferably 1 hour to 24 hours after), or simultaneously with a drug or physical stress. At a specified time (30 minute to 3 days, preferably 1 hour to 2 days, more preferably 1 hour to 24 hours) after administration of the test compound, the amount of the receptor protein of the present invention or its partial peptides contained in cell membranes are quantified.

(ii) Transformants are cultured in a conventional manner and a test compound is added to the culture medium. After a specified time (after 1 day to 7 days, preferably after 1 day to 3 days, more preferably after 2 to 3 days), the amount of the receptor protein of the present invention or its partial peptides contained in the cell membranes can be quantified.

Specifically, the receptor protein of the present invention or its partial peptides contained in cell membrane fractions are confirmed as follows.

(iii) Normal or disease models of non-human mammals (e.g., mice, rats, rabbits, sheep, swine, bovine, cats, dogs, monkeys, etc., more specifically, rats with dementia, obese mice, rabbits with arteriosclerosis, tumor-bearing mice, etc.) receive administration of a drug (e.g., anti-dementia agents, hypotensive agents, anticancer agents, antiobestic agents, etc.) or physical stress (e.g., soaking stress, electric shock, light, darkness, low temperature, etc.), and blood or particular organs (e.g., brain, liver, kidneys, etc.), or tissues or cells isolated from the organs are obtained after a specified period of time. Tissue sections are prepared from the thus obtained organs, tissues, cells, etc. in a conventional manner followed by immunostaining with the antibody of the present invention. The staining intensity of the receptor protein in the cell surface layer is quantified to confirm the protein on the cell membrane, the amount of the receptor protein of the present invention or its partial peptides on the cell membrane can be confirmed quantitatively or qualitatively.

(iv) The confirmation can also be made in a similar manner, using transformants expressing the receptor protein of the present invention or its partial peptides.

The compounds or salts thereof that are obtainable by the screening methods of the present invention are the compounds that alter the amount of the receptor protein or its partial peptide of the present invention. Specifically, these compounds are; (a) compounds that potentiate the G protein-coupled receptor-mediated cell-stimulating activities (e.g., activities that promote or inhibit arachidonic acid release, acetylcholine release, intracellular Ca²⁺ release, intracellular cAMP production, intracellular cGMP production, inositol phosphate production, changes in cell membrane potential, phosphorylation of intracellular proteins, activation of c-fos, pH reduction, etc.), by increasing the amount of the receptor protein of the present invention or its partial peptide on cell membranes; and (b) compounds that reduce the cell stimulating-activities by decreasing the amount of the receptor protein of the present invention or its partial peptide.

The compounds may include peptides, proteins, non-peptide compounds, synthetic compounds, fermentation products, etc., and may be novel or known compounds.

The compounds that potentiate the cell-stimulating activities are useful as safe and low-toxic pharmaceuticals for increasing the physiological activities of the receptor protein or the like of the present invention.

The compounds that decrease the cell-stimulating activities are useful as safe and low-toxic pharmaceuticals for reducing the physiological activities of the receptor protein or the like of the present invention.

When compounds or salts thereof that are obtainable by the screening methods of the present invention are used for pharmaceutical compositions, the compounds can be prepared into pharmaceutical preparations in a conventional manner. For example, as described above for preparing the pharmaceuticals containing the receptor protein of the present invention, the compounds can be prepared in the form of tablets, capsules, elixir, microcapsules, aseptic solution, suspension, etc.

Since the pharmaceutical preparations thus obtained are safe and low toxic, the preparations can be administered, for example, to human or mammals (e.g., rats, rabbits, sheep, swine, bovine, cats, dogs, monkeys, etc.).

The dose of the compounds or salts thereof varies depending on subject to be administered, target organs, symptom, route for administration, etc.; in oral administration, the dose is normally about 0.1 to about 100 mg, preferably about 1.0 to about 50 mg, more preferably about 1.0 to about 20 mg per day for the patient with hypertension (as 60 kg body weight). In parenteral administration, the single dose varies depending on subject to be administered, target organ, symptom, method for administration, etc. but it is advantageous to administer the active ingredient intravenously at a daily dose of about 0.01 to about 30 mg, preferably about 0.1 to about 20 mg, more preferably about 0.1 to about 10 mg for the patient with hypertension (as 60 kg body weight). For other animal species, the corresponding dose as converted per 60 kg weight can be administered.

(11) Prophylactic and/or Therapeutic Agents for Various Diseases Comprising Compounds that Alter the Amount of the Receptor Protein of the Present Invention or its Partial Peptides on Cell Membranes

As described above, the receptor protein of the present invention is considered to play some important role in vivo, such as the central function, etc. Therefore, the compounds that alter the amount of the receptor protein of the present invention or its partial peptide on cell membranes can be used as prophylactic and/or therapeutic agents for diseases associated with dysfunction of the receptor protein of the present invention.

When the compounds are used as prophylactic and/or therapeutic agents for diseases associated with dysfunction of the receptor protein of the present invention, the preparations can be obtained in a conventional manner.

For example, the compounds can be administered orally in the form of tablets which, if necessary, may be sugar coated, capsules, elixir, microcapsules, etc., or parenterally in the form of injectable preparations such as a sterile solution, a suspension, etc. in water or with other pharmaceutically acceptable liquid. For example, these preparations can be manufactured by mixing with physiologically acceptable known carriers, flavors, fillers, vehicles, antiseptics, stabilizers, binders, etc. in a unit dosage form required for generally approved pharmaceutical preparations. The active ingredient in the preparation is controlled in such a dose that an appropriate dose is obtained within the specified range given.

Additives miscible with tablets, capsules etc. include a binder such as gelatin, corn starch, tragacanth and gum arabic, an excipient such as crystalline cellulose, a swelling agent such as corn starch, gelatin, alginic acid, etc., a lubricant such as magnesium stearate, a sweetening agent such as sucrose, lactose and saccharin, and a flavoring agent such as peppermint, akamono oil and cherry. When the unit dosage is in the form of capsules, liquid carriers such as oils and fats may further be used together with the additives described above. A sterile composition for injection may be formulated according to a conventional manner used to make pharmaceutical compositions, e.g., by dissolving or suspending the active ingredients in a vehicle such as water for injection with a naturally occurring vegetable oil such as sesame oil and coconut oil, etc. to prepare the pharmaceutical composition. Examples of an aqueous medium for injection include physiological saline and an isotonic solution containing glucose and other auxiliary agents (e.g., D-sorbitol, D-mannitol, sodium chloride, etc.), etc. and may be used in combination with an appropriate dissolution aid such as an alcohol (e.g., ethanol), a polyalcohol (e.g., propylene glycol and polyethylene glycol), a nonionic surfactant (e.g., polysorbate 80™ and HCO-50), etc. As an oily medium, for example, sesame oil, soybean oil, etc. may be used, which can be used in combination with a dissolution aid such as benzyl benzoate, benzyl alcohol, etc.

Furthermore, the prophylactic/therapeutic agent described above may also be formulated with a buffer (e.g., phosphate buffer and sodium acetate buffer) a soothing agent (e.g., benzalkonium chloride, procaine hydrochloride, etc.), a stabilizer (e.g., human serum albumin, polyethylene glycol, etc.), a preservative (e.g., benzyl alcohol, phenol, etc.), an antioxidant, etc. The thus-prepared liquid for injection is normally filled in an appropriate ampoule.

The pharmaceutical preparations obtained as described above are safe and low toxic, and can be administered to, for example, humans and mammals (e.g., rats, rabbits, sheep, swine, bovine, cats, dogs, monkeys, etc.).

The dose of the compounds or salts thereof varies depending on subject to be administered, target organs, symptom, routes for administration, etc.; in oral administration, the dose is normally about 0.1 to about 100 mg, preferably about 1.0 to about 50 mg, and more preferably about 1.0 to about 20 mg per day for the patient with hypertension (as 60 kg body weight). In parenteral administration, the single dose varies depending on subject to be administered, target organ, symptom, route for administration, etc. but it is advantageous to administer the active ingredient intravenously in a daily dose of about 0.01 to about 30 mg, preferably about 0.1 to about 20 mg, and more preferably about 0.1 to about 10 mg for the patient with hypertension (as 60 kg body weight). For other animal species, the corresponding dose as converted per 60 kg weight can be administered.

(12) Neutralization, with the Antibody to the Receptor Protein of the Present Invention, its Partial Peptide or Salts Thereof

The activity of the antibody to the receptor protein of the present invention its partial peptide or salts thereof that neutralize these receptor protein or the like means the activity of inactivating the signal transduction function, in which the receptor protein takes part. Therefore, when the antibody has the neutralizing activity, the antibody can inactivate the signal transduction in which the receptor protein participates, for example, the receptor protein-mediated cell stimulating activities (e.g., the activities that promote or suppress arachidonic acid release, acetylcholine release, intracellular Ca²⁺ release, intracellular cAMP production, intracellular cGMP production, inositol phosphate production, changes in cell membrane potential, phosphorylation of intracellular proteins, activation of c-fos, pH reduction, etc.). Thus, the antibody can be used for the prevention and/or treatment of diseases caused by overexpression of the receptor protein.

(13) Preparation of Animals Having the DNA Encoding the G Protein-Coupled Receptor Protein of the Present Invention

Using the DNA of the present invention, transgenic animals that express the receptor protein or the like of the present invention can be prepared. Examples of the animals are mammals (e.g., rats, mice, rabbits, sheep, swine, bovine, cats, dogs, monkeys, etc.) or the like (hereinafter sometimes merely referred to as animal) can be used, with particularly preferred being mice, rabbits, etc.

To transfer the DNA of the present invention to a target animal, it is generally advantageous to use the DNA in a gene construct ligated downstream a promoter capable of expressing the DNA in an animal cell. For example, when the rabbit-derived DNA of the present invention is transferred, for example, the gene construct, in which the DNA is ligated downstream a promoter that can expresses the animal-derived DNA of the present invention highly homologous thereto, is microinjected to, e.g., rabbit fertilized ova. Thus, the DNA-transferred animal capable of producing a high level of the receptor protein or the like of the present invention can be prepared. Examples of the promoter that can be used are a virus-derived promoter and a ubiquitous expression promoter such as metallothionein may be used but an NGF gene promoter, an enolase gene promoter, etc. that are specifically expressed in the brain are preferably employed.

The transfer of the DNA of the present invention at the fertilized egg cell stage secures the presence of DNA in all germ and somatic cells in the target animal. The presence of the receptor protein or the like of the present invention in the germ cells in the DNA-transferred animal means that all germ and somatic cells contain the receptor protein or the like of the present invention in all progenies of the animal. The progenies of the animal that took over the gene have the receptor protein or the like of the present invention in all germ and somatic cells.

The transgenic animal to which the DNA of the present invention has been transferred can be subjected to mating and breeding for generations under common breeding circumstance, as the DNA-bearing animal, after confirming that the gene can be stably retained. Moreover, male and female animals having the desired DNA are mated to give a homozygote having the transduced gene in both homologous chromosomes and then the male and female animals are mated so that such breeding for generations that all progenies contain the DNA can be performed.

The transgenic animal to which the DNA of the present invention has been transferred is useful as the animal for screening the agonists or antagonists to the receptor protein or the like of the present invention, since the receptor protein or the like of the present invention is abundantly expressed.

The DNA transgenic animal of the present invention may also be used as the cell sources for tissue culture. The receptor protein or the like of the present invention can be analyzed by, for example, direct analysis of the DNA or RNA in tissues of the DNA-transferred mice of the present invention, or by analysis of tissues containing the receptor protein expressed from the gene. Cells from tissues containing the receptor protein or the like of the present invention are cultured by the standard tissue culture technique. Using these cells the function of the cells from tissues that are generally difficult to culture, for example, cells derived from the brain or peripheral tissues can be studied. Using these cells it is possible to select pharmaceuticals, for example, that increase the functions of various tissues. Where a high expressing cell line is available, the receptor protein or the like of the present invention can be isolated and purified from the cell line.

In the specification and drawings, the codes of bases and amino acids are denoted in accordance with the IUPAC-IUB Commission on Biochemical Nomenclature or by the common codes in the art, examples of which are shown below. For amino acids that may have the optical isomer, L form is shown unless otherwise indicated.

DNA: deoxyribonucleic acid

cDNA: complementary deoxyribonucleic acid

A: adenine

T: thymine

G: guanine

C: cytosine

RNA: ribonucleic acid

mRNA: messenger ribonucleic acid

dATP: deoxyadenosine triphosphate

dTTP: deoxythymidine triphosphate

dGTP: deoxyguanosine triphosphate

dCTP: deoxycytidine triphosphate

ATP: adenosine triphosphate

EDTA: ethylenediarninetetraacetic acid

SDS: sodium dodecyl sulfate

Gly: glycine

Ala: alanine

Val: valine

Leu: leucine

Ile: isoleucine

Ser: serine

Thr: threonine

Cys: cysteine

Met: methionine

Glu: glutamic acid

Asp: aspartic acid

Lys: lysine

Arg: arginine

His: histidine

Phe: phenylalanine

Tyr: tyrosine

Trp: tryptophan

Pro: proline

Asn: asparagine

Gln: glutamine

pGlu: pyroglutamic acid

*: corresponding to termination codon

Me: methyl group

Et: ethyl group

Bu: butyl group

Ph: phenyl group

TC: thiazolidine-4(R)-carboxamide group

Substituents, protecting groups, and reagents generally used in the specification are denoted by the codes shown below.

Tos: p-toluenesulfonyl

CHO: formyl

Bzl: benzyl

Cl₂Bzl: 2,6-dichlorobenzyl

Bom: benzyloxymethyl

Z: benzyloxycarbonyl

Cl-Z: 2-chlorobenzyloxycarbonyl

Br-Z: 2-bromobenzyloxycarbonyl

Boc: t-butoxycarbonyl

DNP: dinitrophenol

Trt: trityl

Bum: t-butoxymethyl

Fmoc: N-9-fluorenylmethoxycarbonyl

HOBt: 1-hydroxybenztriazole

HOOBt: 3,4-dihydro-3-hydroxy-4-oxo-1,2,3-benzotriazine

HONB: 1-hydroxy-5-norbornene-2,3-dicarboximide

DCC: N,N′-dichlorohexylcarbodiimide

The sequence identification numbers in the sequence listing of the specification indicates the following sequence, respectively.

[SEQ ID NO: 1]

This shows the amino acid sequence of human leukocyte-derived novel G protein-coupled receptor protein hTGR3 of the present invention.

[SEQ ID NO: 2]

This shows the base sequence of cDNA encoding human leukocyte-derived novel 6 protein-coupled receptor protein hTGR3 of the present invention.

[SEQ ID NO: 3]

This shows the base sequence of cDNA encoding human leukocyte-derived novel G protein-coupled receptor protein hTGR3 of the present invention.

[SEQ ID NO: 4]

This shows the base sequence of a primer used for cloning the cDNA encoding human leukocyte-derived novel G protein-coupled receptor protein hTGR3 of the present invention.

[SEQ ID NO: 5]

This shows the base sequence of a primer used for cloning the cDNA encoding human leukocyte-derived novel G protein-coupled receptor protein hTGR3 of the present invention.

[SEQ ID NO: 6]

This shows the base sequence of a primer used for analysis of the expression distribution of TGR3 in human tissue, which was performed in EXAMPLE 2.

[SEQ ID NO: 7]

This shows the base sequence of a primer used for analysis of the expression distribution of TGR3 in human tissue, which was performed in EXAMPLE 2.

[SEQ ID NO: 8]

This shows the base sequence of a probe used for analysis of the expression distribution of TGR3 in human tissue, which was performed in EXAMPLE 2.

Transformant Escherichia coli TOP 10/pCR2.1-hTGR3T obtained in EXAMPLE 1 later described has been on deposit with the Ministry of International Trade and Industry, Agency of Industrial Science and Technology, National Institute of Bioscience and Human Technology (NIBH) as the Accession Number FERM BP-7071 since Mar. 6, 2000 and with Institute for Fermentation, Osaka (IFO) as the Accession Number IFO 16358 since Feb. 16, 2000.

Transformant Escherichia coli TOP10/pCR2.1-hTGR3G obtained in EXAMPLE 1 later described has been on deposit with the Ministry of International Trade and Industry, Agency of Industrial Science and Technology, National Institute of Bioscience and Human Technology (NIBH) as the Accession Number FERM BP-7072 since Mar. 6, 2000 and with Institute for Fermentation, Osaka (IFO) as the Accession Number IFO 16359 since Feb. 16, 2000.

Hereinafter, the present invention will be described in more detail with reference to EXAMPLES, but is not intended to limit the scope of the present invention thereto. The gene manipulation procedures using Escherichia coli were carried out in accordance with the methods described in the Molecular Cloning.

### EXAMPLE 1

Cloning of the cDNA Encoding the Human Leukocyte-Derived G Protein-Coupled Receptor Protein and Determination of the Base Sequence

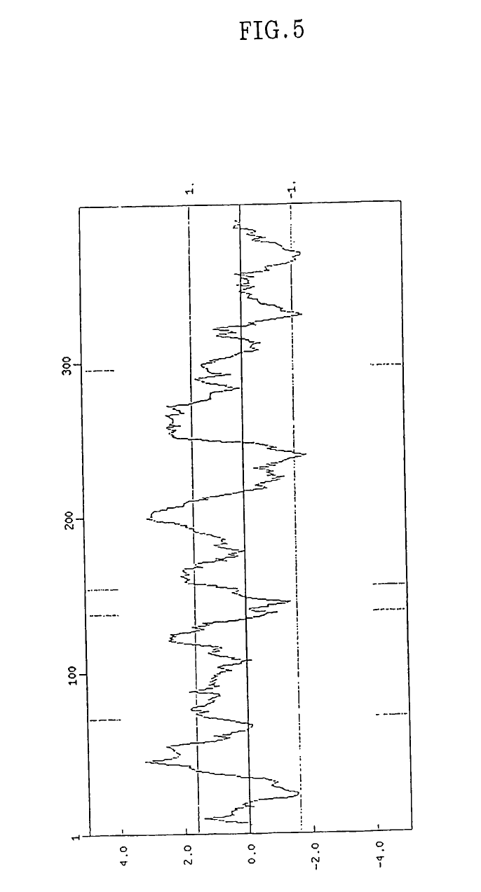

Using human leukocyte cDNA (CLONTECH, Inc.) as the template and using two primers, namely, primer 1 (SEQ ID NO:4) and primer 2 (SEQ ID NO:5), PCR reaction was carried out. The reaction solution in the above reaction was composed of 1/10 volume of the cDNA above as the template, 1/50 volume of TaKaRa LA Taq (TaKaRa K. K.), 0.5 μM each of primer 1 (SEQ ID NO:4) and primer 2 (SEQ ID NO:5), 200 μM of dNTPs and ½ volume of GC Buffer attached to the enzyme to make the total volume 20 μl. In the PCR reaction, the reaction at 94° C. for 5 minutes was followed by 35 repetitions of the cycle set at 94° C. for 30 seconds and then at 68° C. for 2 minutes, and finally, extension reaction was performed at 68° C. for 5 minutes. The PCR product was subcloned to plasmid vector pCR2.1 (Invitrogen Inc.) following the instructions attached to TA cloning kit (Invitrogen Inc.), which was then introduced into Escherichia coli TOP 10, and the clones carrying the cDNA were selected on LB agar medium containing ampicillin. The sequence of each clone was analyzed to give the cDNA sequences (SEQ ID NOs:2 and 3) encoding the novel G protein-coupled receptor protein. These two sequences were different at the 621st residue by one base, but the amino acid sequence deduced therefrom was the amino acid sequence shown by SEQ ID NO: 1. The novel G protein-coupled receptor protein having this amino acid sequence was named hTGR3. The two transformants were named Escherichia coli TP10/pCR2.1-hTGR3T and Escherichia coli TP10/pCR2.1-hTGR3G, respectively. The hydrophobic plot of hTGR3 is also shown in FIG. 5.

### EXAMPLE 2

Analysis on the Expression Distribution of TGR3 in Human Tissues

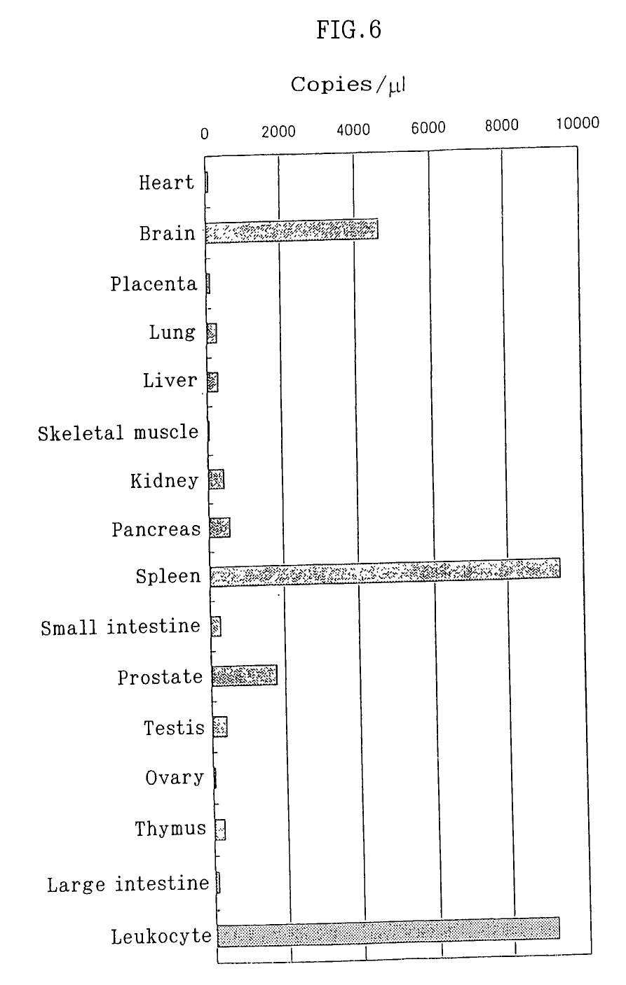

The expression distribution of TGR3 in human tissues was analyzed using the TaqMan PCR method. Using Human Multiple Tissue cDNA Panel (CLONTECH, Inc.) as a template, TaqMan PCR was carried out using as primers for the PCR primer 3 (SEQ ID NO: 6 (TGGACGCTTGCTCCACTGT)) and primer 4 (SEQ ID NO:7 (AGCACGCAGAAGAGCACGT)) and a probe having the base sequence shown by SEQ ID NO: 8 (TTGCCGCTCTACGCCAAGGCC). The reaction solution in the reaction contained 12.5 μl of TaqMan Universal PCR Master Mix (Applied Biosystems Japan), 0.5 μl each of 10 μM primer 1 and primer 2, 1 μl of 5 μM probe and 2 μl of the template, and 8.5 μl of distilled water was added to make the total volume 25 μl. PCR was performed by maintaining at 50° C. for 2 minutes and 95° C. for 10 minutes, followed by 40 repetitions of the cycle set to include 95° C. for 15 seconds and 60° C. for 1 minute. Based on the results obtained, the number of copies per 1 μl of the cDNA was calculated, which results are shown in FIG. 6. The results reveal that the expression level of TGR3 was high in the spleen, leukocyte and brain.

### INDUSTRIAL APPLICABILITY

The G protein-coupled receptor protein of the present invention, its partial peptide or salts thereof as well as the polynucleotide encoding the receptor protein or its partial peptide (e.g., DNA, RNA and derivatives thereof) can be used: (1) for determination of ligands (agonists), (2) for preparing the antibodies and antisera, (3) for construction of the expression system of a recombinant receptor protein, (4) for development of the receptor-binding assay system using the expression system and screening of candidate pharmaceutical compounds, (5) for drug design based on the comparison with structurally similar ligands-receptors, (6) as reagents for preparing probes or PCR primers in gene diagnosis, (7) for preparing a transgenic animal, or (8) as pharmaceuticals such as prophylactic/therapeutic agents in gene therapy, etc.

1. A G protein-coupled receptor protein containing the same or substantially the same amino acid sequence as the amino acid sequence shown by SEQ ID NO: 1, or a salt thereof.

2. A partial peptide of the G protein-coupled receptor protein according to claim 1, or a salt thereof.

3. A polynucleotide containing a polynucleotide encoding the G protein-coupled receptor protein according to claim 1 or the partial peptide according to claim 2.

4. The polynucleotide according to claim 4, which is a DNA.

5. The polynucleotide according to claim 3, which has the base sequence represented by SEQ ID NO: 2 or SEQ ID NO: 3.

6. A recombinant vector containing the polynucleotide according to claim 3.

7. A transformant transformed by the recombinant vector according to claim 6.

8. A method of manufacturing the G protein-coupled receptor protein according to claim 1 or the partial peptide according to claim 2, or a salt thereof, which comprises culturing the transformant according to claim 7 and producing the G protein-coupled receptor protein according to claim 1 or the partial peptide according to claim 2.

9. An antibody to the G protein-coupled receptor protein according to claim 1 or the partial peptide according to claim 2, or a salt thereof.

10. The Antibody according to claim 9, which is a neutralizing antibody to inactivate signal transduction of the G protein-coupled receptor protein according to claim 1.

11. A diagnostic composition comprising the antibody according to claim 9.

12. A ligand to the G protein-coupled receptor protein or its salt according to claim 1, which is obtainable using the G protein-coupled receptor protein according to claim 1 or the partial peptide according to claim 2, or a salt thereof.

13. A pharmaceutical composition comprising the ligand to the G protein-coupled receptor protein according to claim 12.

14. A method of determining a ligand to the G protein-coupled receptor protein or its salt according to claim 1, wherein the G protein-coupled receptor protein according to claim 1 or the partial peptide according to claim 2, or a salt thereof is used.

15. A method of screening a compound or its salt that alters the binding property between a ligand and the G protein-coupled receptor protein or its salt according to claim 1, which comprises using the G protein-coupled receptor protein according to claim 1 or the partial peptide according to claim 2, or a salt thereof.

16. A kit for screening a compound or its salt that alters the binding property between a ligand and the G protein-coupled receptor protein or its salt according to claim 1, comprising the G protein-coupled receptor protein according to claim 1 or the partial peptide according to claim 2, or a salt thereof.

17. A compound or its salt that alters the binding property between a ligand and the G protein-coupled receptor protein or its salt according to claim 1, which is obtainable using the screening method according to claim 15 or the screening kit according to claim 16.

18. A pharmaceutical composition comprising the compound or its salt that alters the binding property between a ligand and the G protein-coupled receptor protein or its salt according to claim 1, which is obtainable using the screening method according to claim 15 or the screening kit according to claim 16.

19. A polynucleotide hybridizable to the polynucleotide according to claim 3 under high stringent conditions.

20. A polynucleotide comprising a base sequence complementary to the polynucleotide according to claim 3, or a part thereof.

21. A method of quantifying mRNA of the G protein-coupled receptor protein according to claim 1, which comprises using the polynucleotide according to claim 3, or a part thereof.

22. A method of quantifying the G protein-coupled receptor protein according to claim 1, which comprises using the antibody according to claim 9.

23. A diagnostic agent for diseases associated with the function of the G protein-coupled receptor protein according to claim 1, which comprises using the quantifying method according to claim 21 or 22.

24. A method of screening a compound or its salt that alters the expression level of the G protein-coupled receptor protein according to claim 1, which comprises using the quantifying method according to claim 21.

25. A method of screening a compound or its salt that alters the amount of the G protein-coupled receptor protein according to claim 1 on a cell membrane, which comprises using the quantifying method according to claim 22.

26. A compound or its salt that alters the expression level of the G protein-coupled receptor protein according to claim 1, which is obtainable using the screening method according to claim 24.

27. A compound or its salt that alters the amount of the G protein-coupled receptor protein according to claim 1 on a cell membrane, which is obtainable using the screening method according to claim 25.


SEQUENCE LISTING (8 sequences)

SEQ ID NO: 1 - protein, 398 aa, Human
MESGLLRPAPVSEVIVLHYNYTGKLRGARYQPGAGLRADAVVCLAVCAFIVLENLAVLLVLGRHPRFHAPMFLLLGSLTLSDLLAGAAYAANILLSGPLTLKLSPALWFAREGGVFVALTASVLSLLAIALERSLTMARRGPAPVSSRGRTLAMAAAAWGVSLLLGLLPALGWNCLGRLDACSTVLPLYAKAYVLFCVLAFVGILAAICALYARIYCQVRANARRLPARPGTAGTTSTRARRKPRSLALLRTLSVVLLAFVACWGPLFLLLLLDVACPARTCPVLLQADPFLGLAMANSLLNPIIYTLTNRDLRHALLRLVCCGRHSCGRDPSGSQQSASAAEASGGLRRCLPPGLDGSFSGSERSSPQRDGLDTSGSTGSPGAPTAARTLVSEPAAD

SEQ ID NO: 2 - DNA, 1194 bp, Human
atggagtcggggctgctgcggccggcgccggtgagcgaggtcatcgtcctgcattacaactacaccggcaagctccgcggtgcgcgctaccagccgggtgccggcctgcgcgccgacgccgtggtgtgcctggcggtgtgcgccttcatcgtgctagagaatctagccgtgttgttggtgctcggacgccacccgcgcttccacgctcccatgttcctgctcctgggcagcctcacgttgtcggatctgctggcaggcgccgcctacgccgccaacatcctactgtcggggccgctcacgctgaaactgtcccccgcgctctggttcgcacgggagggaggcgtcttcgtggcactcactgcgtccgtgctgagcctcctggccatcgcgctggagcgcagcctcaccatggcgcgcagggggcccgcgcccgtctccagtcgggggcgcacgctggcgatggcagccgcggcctggggcgtgtcgctgctcctcgggctcctgccagcgctgggctggaattgcctgggtcgcctggacgcttgctccactgtcttgccgctctacgccaaggcctacgtgctcttctgcgtgctcgccttcgtgggcatcctggccgctatctgtgcactctacgcgcgcatctactgccaggtacgcgccaacgcgcggcgcctgccggcacggcccgggactgcggggaccacctcgacccgggcgcgtcgcaagccgcgctcgctggccttgctgcgcacgctcagcgtggtgctcctggcctttgtggcatgttggggccccctcttcctgctgctgttgctcgacgtggcgtgcccggcgcgcacctgtcctgtactcctgcaggccgatcccttcctgggactggccatggccaactcacttctgaaccccatcatctacacgctcaccaaccgcgacctgcgccacgcgctcctgcgcctggtctgctgcggacgccactcctgcggcagagacccgagtggctcccagcagtcggcgagcgcggctgaggcttccgggggcctgcgccgctgcctgcccccgggccttgatgggagcttcagcggctcggagcgctcatcgccccagcgcgacgggctggacaccagcggctccacaggcagccccggtgcacccacagccgcccggactctggtatcagaaccggctgcagac

SEQ ID NO: 3 - DNA, 1194 bp, Human
atggagtcggggctgctgcggccggcgccggtgagcgaggtcatcgtcctgcattacaactacaccggcaagctccgcggtgcgcgctaccagccgggtgccggcctgcgcgccgacgccgtggtgtgcctggcggtgtgcgccttcatcgtgctagagaatctagccgtgttgttggtgctcggacgccacccgcgcttccacgctcccatgttcctgctcctgggcagcctcacgttgtcggatctgctggcaggcgccgcctacgccgccaacatcctactgtcggggccgctcacgctgaaactgtcccccgcgctctggttcgcacgggagggaggcgtcttcgtggcactcactgcgtccgtgctgagcctcctggccatcgcgctggagcgcagcctcaccatggcgcgcagggggcccgcgcccgtctccagtcgggggcgcacgctggcgatggcagccgcggcctggggcgtgtcgctgctcctcgggctcctgccagcgctgggctggaattgcctgggtcgcctggacgcttgctccactgtcttgccgctctacgccaaggcctacgtgctcttctgcgtgctcgccttcgtgggcatcctggccgcgatctgtgcactctacgcgcgcatctactgccaggtacgcgccaacgcgcggcgcctgccggcacggcccgggactgcggggaccacctcgacccgggcgcgtcgcaagccgcgctcgctggccttgctgcgcacgctcagcgtggtgctcctggcctttgtggcatgttggggccccctcttcctgctgctgttgctcgacgtggcgtgcccggcgcgcacctgtcctgtactcctgcaggccgatcccttcctgggactggccatggccaactcacttctgaaccccatcatctacacgctcaccaaccgcgacctgcgccacgcgctcctgcgcctggtctgctgcggacgccactcctgcggcagagacccgagtggctcccagcagtcggcgagcgcggctgaggcttccgggggcctgcgccgctgcctgcccccgggccttgatgggagcttcagcggctcggagcgctcatcgccccagcgcgacgggctggacaccagcggctccacaggcagccccggtgcacccacagccgcccggactctggtatcagaaccggctgcagac

SEQ ID NO: 4 - DNA, 36 bp, Artificial Sequence
  Primer
gtcgacatggagtcggggctgctgcggccggcgccg

SEQ ID NO: 5 - DNA, 36 bp, Artificial Sequence
  Primer
tactagttcagtctgcagccggttctgataccagag

SEQ ID NO: 6 - DNA, 19 bp, Artificial Sequence
  Primer
tggacgcttgctccactgt

SEQ ID NO: 7 - DNA, 19 bp, Artificial Sequence
  Primer
agcacgcagaagagcacgt

SEQ ID NO: 8 - DNA, 21 bp, Artificial Sequence
  Probe
ttgccgctctacgccaaggcc